(19) United States
(12) Patent Application Publication
(10) Pub. No.: US 2019/0134330 A1
(43) Pub. Date: May 9, 2019

(54) SYSTEMS AND METHODS FOR PULMONARY HEALTH MANAGEMENT

(71) Applicant: PNEUMA RESPIRATOR, INC., Boone, NC (US)

(72) Inventors: Louis Thomas Germinario, Kingsport, TN (US); John H. Hebrank, Durham, NC (US); Charles Eric Hunter, Boone, NC (US); Thomas P. Stern, Huntersville, NC (US)

(73) Assignee: PNEUMA RESPIRATOR, INC., Boone, NC (US)

(21) Appl. No.: 16/098,806

(22) PCT Filed: May 3, 2017
(86) PCT No.: PCT/US17/30925

§ 371 Date: Nov. 2, 2018

Related U.S. Application Data
(60) Provisional application No. 62/331,328, filed on May 3, 2016.
(60) Provisional application No. 62/332,352, filed on May 5, 2016.
(60) Provisional application No. 62/334,076, filed on May 10, 2016.
(60) Provisional application No. 62/354,437, filed on Jun. 24, 2016.
(60) Provisional application No. 62/399,091, filed on Sep. 23, 2016.
(60) Provisional application No. 62/416,026, filed on Nov. 1, 2016.
(60) Provisional application No. 62/422,932, filed on Nov. 16, 2016.
(60) Provisional application No. 62/428,696, filed on Dec. 1, 2016.
(60) Provisional application No. 62/448,796, filed on Jan. 20, 2017.
(60) Provisional application No. 62/471,929, filed on Mar. 15, 2017.

(51) Int. Cl. A61M 16/00; A61M 15/00; A61M 11/00; G16H 50/20

(52) U.S. Cl. CPC A61M 16/024; A61M 15/0021; A61M 11/003; A61M 16/0051; A61M 15/0085; G16H 50/20; A61M 2205/502; A61M 2016/0039; A61M 2016/0021; A61M 2205/3327; A61M 2205/3306; A61M 2205/3553; A61M 2205/3584; A61M 2016/0027

(57) ABSTRACT

The present disclosure provides system and methods for pulmonary health using one or more inhalation devices. In one aspect, the air inhalation devices each comprise one or more sensors configured to capture pulmonary health data for a patient. Using this data, air analytics may be generated pertaining to individualized patient health, general health for people living within a particular geographical location, air quality for a particular geographical region, operational parameters of the inhalation devices, and/or the like. The air analytics may be output, for example, for display on a user device, such as a patient user device, a health care provider user device, and/or an administrator user device.

### CROSS-REFERENCE TO RELATED APPLICATIONS

The present application claims benefit under 35 U.S.C. § 119 of: U.S. Provisional Patent Application No. 62/331,328, entitled “DISPOSABLE PULMONARY DRUG DELIVERY APPARATUS AND METHODS OF USE,” filed on May 3, 2016; U.S. Provisional Patent Application No. 62/332,352, entitled “DISPOSABLE PULMONARY DRUG DELIVERY APPARATUS AND METHODS OF USE,” filed on May 5, 2016; U.S. Provisional Patent Application No. 62/334,076, entitled “DISPOSABLE PULMONARY DRUG DELIVERY APPARATUS AND METHODS OF USE,” filed on May 10, 2016; U.S. Provisional Patent Application No. 62/354,437, entitled “DISPOSABLE PULMONARY DRUG DELIVERY APPARATUS AND METHODS OF USE,” filed on Jun. 24, 2016; U.S. Provisional Patent Application No. 62/399,091, entitled “DISPOSABLE PULMONARY DRUG DELIVERY APPARATUS AND METHODS OF USE,” filed on Sep. 23, 2016; U.S. Provisional Patent Application No. 62/416,026, entitled “DISPOSABLE PULMONARY DRUG DELIVERY APPARATUS AND METHODS OF USE,” filed on Nov. 1, 2016; U.S. Provisional Patent Application No. 62/422,932, entitled “DISPOSABLE PULMONARY DRUG DELIVERY APPARATUS AND METHODS OF USE,” filed on Nov. 16, 2016; U.S. Provisional Patent Application No. 62/428,696, entitled “DISPOSABLE PULMONARY DRUG DELIVERY APPARATUS AND METHODS OF USE,” filed on Dec. 1, 2016; U.S. Provisional Patent Application No. 62/448,796, entitled “DISPOSABLE PULMONARY DRUG DELIVERY APPARATUS AND METHODS OF USE,” filed on Jan. 20, 2017; and U.S. Provisional Patent Application No. 62/471,929, entitled “DISPOSABLE PULMONARY DRUG DELIVERY APPARATUS AND METHODS OF USE,” filed on Mar. 15, 2017. The content of each application is incorporated herein by reference in its entirety.

### TECHNICAL FIELD

Aspects of the present disclosure involve pulmonary health management for one or more patients and more particularly to generating pulmonary health management analytics using one or more inhalation devices configured to monitor pulmonary conditions and deliver drugs through the lungs to treat a variety of pulmonary and/or non-pulmonary conditions.

### BACKGROUND

A variety of respiratory diseases are often treated using aerosol generating devices. For example, inhalation provides for the delivery of aerosolized drugs to treat asthma, chronic obstructive pulmonary disease (COPD), and site-specific conditions, with reduced systemic adverse effects. A major challenge is providing a device that delivers an accurate, consistent, and verifiable dose, with a droplet size that is suitable for successful delivery to the targeted lung passageways. Effective delivery of medication to the deep pulmonary regions of the lungs through the alveoli, has always posed a problem, especially to children and elderly, as well as to those with a diseased state, owing to their limited lung capacity and constriction of the breathing passageways. The impact of constricted lung passageways limits deep inspiration and synchronization of the administered dose with a breathing cycle, including inspiration and expiration, of the patient.

Conventional devices often either fail to impart a momentum sufficiently high to achieve ejection of the dose from the device or sufficiently low to prevent deposition of the dose on the back of the throat. More particularly, many conventional inhaler systems, such as metered dose inhalers (MDI) and pressurized metered dose inhalers (p-MDI) or pneumatic and ultrasonic-driven devices, generally produce drops with high velocities and a wide range of droplet sizes including large droplets that have high momentum and kinetic energy. Droplets and aerosols with such high momentum do not reach the distal lung or lower pulmonary passageways but are instead deposited in the mouth and throat. As a result, larger total drug doses are required to achieve the desired deposition in targeted areas. These large doses increase the probability of unwanted side effects.

Additional challenges arise where aerosol plumes generated from conventional aerosol delivery systems, as a result of their high ejection velocities and the rapid expansion of the drug carrying propellant, lead to localized cooling and subsequent condensation, deposition and crystallization of drug onto the ejector surfaces. Such blockage of ejector apertures by deposited drug residue may inhibit the effective delivery of a dose. This phenomenon of surface condensation and drug deposition is also a challenge for conventional nebulizers.

Further, many patients fail to use inhalers correctly, exacerbating the challenges to health care providers in treating various conditions. Delivery and inhalation of the correct dose at prescribed times is important to treatment. However, conventional devices fail to verify the delivery and quality of a dose, leaving providers struggling to interpret the current treatment results and revise a prescribed treatment when the therapeutic result is not obtained. Similarly, providers have to rely on a description of symptoms by the patient in combination with clinical tests. However, this often fails to provide a comprehensive picture of the condition and the effectiveness of a particular drug therapy in treating a pulmonary condition.

It is with these observations in mind, among others, that various aspects of the present disclosure were conceived and developed.

### SUMMARY

Implementations described and claimed herein address the foregoing problems by providing systems and methods for pulmonary health management. In one implementation, pulmonary health management information is received from one or more inhalation devices over a network. Each of the one or more inhalation devices has one or more pressure sensors measuring a flow rate of an air flow through a tube of the inhalation device. Environmental data is received for one or more geographical locations in which the one or more inhalation devices are deployed. The environmental data is captured using one or more environmental sensors and corresponds to an ambient air condition of each of the one or more geographical locations. The pulmonary health management information is correlated with the environmental data based on at least one management parameter using at least one computing unit; and air analytics are generated from the correlated data using the at least one computing unit.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

FIG. 1 illustrates an example inhalation device used by a patient for pulmonary health management.

FIG. 2 is a functional block diagram of example components of the inhalation device, including a drug cartridge.

FIG. 3 shows example operations for medication delivery for a patient using an inhalation device.

FIG. 4 illustrates a pulmonary health management system, including an air analyzer which may run on a computer server, computing device, or other network device, for pulmonary health management using one or more inhalation devices.

FIG. 5 is an example air analytics user interface for managing pulmonary health of one or more patients or a geographical area.

FIG. 6 illustrates example operations for pulmonary health management.

FIG. 7 is a functional block diagram of an electronic device including operational units arranged to perform pulmonary health management operations.

FIG. 8 is an example computing system that may implement various systems and methods of the presently disclosed technology.

FIGS. 9A-9C show a front perspective, back perspective, and back view, respectively, of an example inhalation device.

FIG. 10 is a cross-sectional view of the inhalation device taken along the line shown in FIG. 9C.

FIG. 11 shows an exploded view of the inhalation device.

FIG. 12A illustrates a bottom perspective view of an example drug cartridge.

FIG. 12B is an exploded view of an ejector assembly of the drug cartridge.

FIG. 12C illustrates a cross-sectional view of the drug cartridge with a detailed view of an ejector o-ring.

FIG. 13 shows detailed views of an example aperature plate with ejector holes having a plurality of diameters configured to generate droplets of a plurality of sizes to target different regions of the pulmonary airways.

FIGS. 14A-14B each show an example inhalation device configured to sense airflow by detecting pressure differentials across flow restriction, with FIG. 14A showing the restriction internal to an aerosol delivery tube and FIG. 14B showing the restriction being an air inlet screen.

FIGS. 15A-15C show various examples of a pressure sensor assembly.

FIG. 16A is a circuit diagram for an example power supply on/off circuit for an example inhalation device.

FIG. 16B is a circuit diagram for an example power supply connector circuit for an example inhalation device.

FIG. 16C is a circuit diagram for an example volt power conditioning circuit for an example inhalation device.

FIG. 16D is a circuit diagram for an example controller circuit for an example inhalation device.

FIG. 16E is a circuit diagram for an example controller programming connector circuit for an example inhalation device.

FIG. 16F is a circuit diagram for an example piezo connector circuit for an example inhalation device.

FIG. 16G is a circuit diagram for an example user switch with debounce circuit for an example inhalation device.

FIG. 16H is a circuit diagram for an example piezo driver circuit for an example inhalation device.

FIG. 16I is a circuit diagram for an example pressure sensor circuit for an example inhalation device.

FIG. 16J is a circuit diagram for an example light emitting diode indicator circuit for an example inhalation device.

FIG. 16K is a circuit diagram for an example buzzer driver circuit for an example inhalation device.

### DETAILED DESCRIPTION

Aspects of the present disclosure generally relate to system and methods for pulmonary health using one or more inhalation devices. In one aspect, the air inhalation devices each comprise one or more sensors configured to capture pulmonary health data for a patient. Using this data, air analytics may be generated pertaining to individualized patient health, general health for people living within a particular geographical location, air quality for a particular geographical region, operational parameters of the inhalation devices, and/or the like. The air analytics may be output, for example, for display on a user device, such as a patient user device, a health care provider user device, and/or an administrator user device.

Generally, the inhalation devices provide delivery of fluid in the form of an aerosol including one or more medicaments to the lungs of a patient for local pulmonary and/or systemic delivery of the medicament(s). The inhalation devices are configured to provide aerosol droplet sizes within the human respirable range such that effective, repeatable dose delivery is achieved. The human respirable range includes droplets with aerodynamic diameters less than 5 μm, which are transported almost completely by motion of the airstream and entrained air, rather than by their own momentum. More particularly, for optimum deposition in alveolar airways, the human respirable range includes droplets with aerodynamic diameters in the ranges of 1 to 5 μm, with particles below 4 μm reaching the alveolar region of the lungs. Larger droplets (i.e., great than 5 μm) are often deposited on the tongue or strike the throat and coat the bronchial passages. Smaller particles, less than 1 μm in diameter, that penetrate more deeply into the lungs have a tendency to be exhaled. Accordingly, the inhaler devices described herein deliver particles of a suitable size range, avoid surface fluid deposition and blockage of apertures, while providing a dose that is verifiable. As such feedback regarding correct and consistent usage of the inhaler device is provided to the patient and the health care provider.

In addition, the inhaler devices may monitor the breathing pattern of the patient for diagnosing a condition and/or managing treatment. For example, a drop in peak flow is a useful indicator for the increasing probability of an asthma attack, and measurement of pulmonary output (i.e. FEV1) can be useful in determining the effectiveness of a particular drug therapy in treating a pulmonary condition.

Many of the example implementations described herein reference pulmonary conditions. However, it will be appreciated by those skilled in the art that the presently disclosed technology is applicable to non-pulmonary conditions that may be diagnosed and/or treated via analysis of and delivery of medication via the lungs. Further, the various inhalation devices described herein are exemplary only. Other health monitoring devices may be used to generate air analytics.

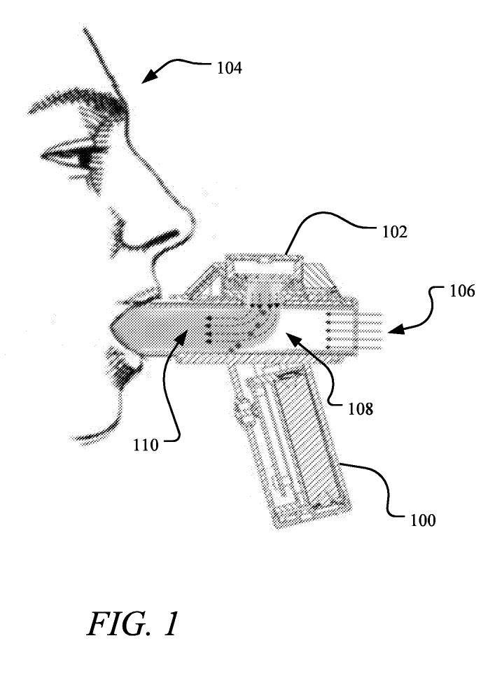

Turning first to FIG. 1, an example inhalation device 100 used by a patient 104 for pulmonary health management is shown. In one implementation, the inhalation device 100 includes a cartridge 102, which may be disposable or reusable, for ejecting a dose of medication for delivery to the lungs of the patient 104 during an inhalation cycle. The inhalation device 100 may further capture pulmonary health data for the patient 104 for use in diagnosing a condition of the patient and/or generating air analytics, as described herein.

In one implementation, the inhalation device 100 is configured for electronic activated breath actuation. The inhalation device 100 includes a pressure sensor assembly that automatically detects a trigger point during an inhalation cycle of the patient 104 to activate and trigger the spray and delivery of medication. For example, the pressure sensor assembly may be programmed to trigger a two second spray when the airflow is ten standard liters per minute (SLM). The trigger point during the inspiratory cycle may provide an optimum point during the inhalation cycle of the patient 104 to activate and trigger the spray and delivery of medication. Since breath actuation does not require patient-device coordination, the breath actuated triggering mechanism of the inhalation device 100 for delivering an aerosol dose ensures optimum delivery of medication.

As shown in FIG. 1, the inhalation device 100 generates entrained air from droplets ejected from the cartridge 102 as a consequence of the combined momentum transfer from the droplets to the surrounding air and the large specific surface area of droplet particles (e.g., 5 μm and less in diameter). FIG. 1 shows the inertial filter mechanism provided by the inhalation device 100 for filtering and excluding larger droplets (e.g., greater than 5 μm in diameter) from an aerosol plume by virtue of their higher inertial force and momentum. In one implementation, the droplets undergo an approximately ninety degree change in spray direction from a first region 108 to a second region 110 as droplets emerge from the cartridge 102 and are swept by the airflow 106 through the laminar flow elements before inhalation into the pulmonary airways of the patient 104.

In the event that droplet particles having an aerodynamic diameter larger than 5 microns are generated by the cartridge 102, the inhalation device 100 excludes them from the airflow 106 by depositing them onto the airway tube using their increased inertial mass. Alternatively, if all ejected particles are targeted for delivery as a dose, a velocity of the airflow 106 may be increased so larger droplet particles may be carried into the pulmonary airways of the patient 104. This feature of the inhalation device 100 further increases the respirable dose, thus providing an improved targeted delivery of medication to either deep into the lower alveolar airways or onto upper respiratory regions of the patient 104.

The inhalation device 100 may further validate a dose through a verification of a presence of a plume of drug droplets. In one implementation, the inhalation device 100 verifies the plume through a measurement of optical absorption or scattering by the droplets between the cartridge 102 and the patient 104. The optical measurements made with a solar-blind ultraviolet light source and detector. In another implementation, the inhalation device 100 verifies the plume by measuring a temperature of the airstream carrying the plume.

As further described herein, in one implementation, the inhalation device 100 generates diagnostics for pulmonary disease state monitoring using an air flow sensor assembly having increased sensitivity at low differential pressures, drift free, low hysteresis, and sensor output that is independent of air density and temperature. More particularly, the inhalation device 100 may capture one or more diagnostic indicators of lung function of the patient 104, including, but not limited to, peak inhalation flow rate, inhalation volume, nitric oxide concentration, pulmonary output, and/or the like. The inhalation device 100 may capture the diagnostic indicators by measurement of a pressure drop between an inhalation tube of the inhalation device 100 and the surrounding atmosphere using a preset flow resistance element, an adjustable flow resistance element, and/or the like. The diagnostic indicators may be stored in the inhalation device 100 and/or communicated to a user device for generating air analytics.

Generally, the inhalation device 100 includes the cartridge 102 for delivering safe, suitable, and repeatable dosages to the patient 104 for pulmonary use. The cartridge 102 delivers a defined volume of fluid in the form of aerosol droplets having properties that deliver an adequate and repeatable high percentage deposition into the alveolar airways of the patient 104 upon application.

As described in more detail herein, in one implementation, the cartridge 102 includes a reservoir for receiving a volume of fluid and an ejector assembly, having a piezoelectric actuator plate and aperture plate (mesh plate) constructed to eject a stream of aerosol droplets having an average ejected droplet diameter of less than 5 microns. Aerosol droplets that are this small, have a low inertial force and low momentum, and are transported almost completely by motion of an air stream, (entrained air), into the lungs, and reach the lower alveolar airways of the subject during use.

Stated differently, the inhalation device 100 delivers an aerosol to the lungs of the patient 104 for the delivery of small molecule, large molecule, and biologic medicaments for local pulmonary or systemic drug delivery. In one implementation, the inhalation device 100 provides aerosol droplet sizes within the human respirable range (e.g., less than 5 microns) such that effective, repeatable dose delivery is achieved. Further, the inhalation device 100 generates aerosols in a manner that does not generate elevated in-situ temperatures or forces that may tend to denature or decompose active agents. As such, the inhalation device 100 may be used to deliver biologics and other large molecules that might otherwise be susceptible to denaturing and degradation in traditional inhalers and nebulizers.

In one implementation, the cartridge 102 includes an ejector assembly constructed to eject the aerosol stream of droplets. The ejector assembly includes an aperture plate that is coupled to a piezoelectric actuator. In certain implementations, the aperture plate may be coupled to an actuator plate that is coupled to the piezoelectric actuator. The aperture plate contains a plurality of openings formed through its thickness and the piezoelectric actuator oscillates the aperture plate, having fluid in contact with one surface of the aperture plate, at a frequency and voltage to generate a directed aerosol stream of droplets through the smaller openings of the aperture plate into the lungs, as the patient 104 inhales. In other implementations where the aperture plate is coupled to the actuator plate, the actuator plate is oscillated by the piezoelectric oscillator at a frequency and voltage to generate a directed aerosol stream or plume of aerosol droplets.

The cartridge 102 can be replaced or disposed either on a daily or weekly or monthly basis suitable to the prescribed treatment. The disposability of the cartridge 102 may minimize and prevent buildup of surface deposits or surface microbial contamination on the aperture plate, owing to its short in-use time. Alternatively, the cartridge 102 may be reusable for delivering multiple medications or flavors of a drug for polypharmacy. Thus, the inhalation device 100 can deliver multiple medications prescribed to a patient and delivered through the same device. The cartridge 102 may include dual drug cartridge module having aperture plates that are similar in design and able to deliver doses of inhaled medications that are targeted for similar regions of the pulmonary airways and have droplets with similar aerodynamic particle size distributions. Alternatively, use of multiple medications or polypharmacy, may require delivery of the medication to different areas of the pulmonary airway. Under these circumstances, each of the dual cartridge modules of the cartridge 102 may have aperture plates with different ejector hole designs and different entrance and exit orifice size to deliver different droplet sizes targeting different regions of the pulmonary airways of the patient 104.

In one implementation, the cartridge 102 delivers multiple medications or flavors of a drug for polypharmacy in which the ejector aperture plate of each drug-containing cartridge unit has a design with different size ejector holes. This design provides a system and method for creating different droplet sizes using one aperture plate. This design may target different regions of the pulmonary airways and thereby facilitate compliance and adherence to medication regimens by children and adults by providing flavored medications which improve the taste of the medication in their mouths and throats. In certain aspects, this system and method of designing aperture plates with different size ejector holes may improve the taste or feel of nicotine delivery devices by delivering droplets that are sufficiently large to deposit onto the tongue and throat as well as delivering droplets into the deep alveolar airways.

The ejector assembly of the cartridge 102 may be horizontally oriented and positioned such that the fluid or drugs contained therein are in constant contact with the entrance surface of the aperture plate. The horizontally oriented ejector assembly allows and provides a uniform distribution of fluid and a uniform coating of fluid onto the aperture plate. In some implementations, the horizontally positioned ejector assembly provides an aperture plate with a uniform fluid/drug coating that also has the benefit of providing a uniform load across the aperture plate. This design provides a more efficient and stable aperture plate oscillation and may provide for a more efficient ejection of fluid and minimizes the probability of chaotic membrane oscillations. In certain aspects, chaotic oscillations may lead to delivery of improper dosages as well as minimize or stop ejection altogether or lead to deposition of fluid and/or drug onto the aperture plate surfaces and lead to blockage of apertures. The reduction or elimination of chaotic oscillations provide a more efficient and stable aperture plate oscillation and a more efficient and stable delivery of medication.

In one implementation, the inhalation device 100 monitors and detects the amount of fluid ejected and medication remaining in the cartridge 102 after a dose is ejected from the piezoelectric ejector of the ejection assembly. A frequency shift in the resonance frequency oscillation of the aperture plate is associated with a change in mass loading, as described by the Sauerbrey equation, for example. In certain implementations, the detection of a change in resonant frequency of oscillation of the aperture plate may provide for dose verification since a change in the resonant frequency of oscillation of the aperture plate is correlated with a change in mass or loading on the piezoelectric actuator.

The inhalation device 100 may include surface tension plate, such as a porous grid or perforated plate. The surface tension plate may include a collection of vertices, edges and holes that define a shape, which may be, without limitation, square, triangular, polygonal, or any shape grid form that is placed behind the aperture plate on the fluid entrance surface-side and configured to maintain a constant fluid loading. The space of the openings on the surface tension plate, as well as the distance and placement of the surface tension plate with respect to the aperture plate surface, may be optimized and specified by the surface tension of the fluid. In certain aspects, the presence of a surface tension plate placed behind the aperture plate also leads to a more stable and controlled oscillation and actuation of the aperture plate and minimize chaotic oscillations and deposition of fluid and/or drug onto the aperture plate surfaces.

In one implementation, the ejector assembly of the cartridge 102 may be vertically oriented, such that the placement of a surface tension plate behind the vertically oriented ejector assembly on the fluid side of the aperture plate provides a constant and uniform fluid loading on the aperture plate. This arrangement provides a uniform fluid coating on the aperture plate and also has the benefit of providing a uniform load across the aperture plate. In certain implementations, this arrangement also provides a more efficient and stable aperture plate oscillation that may lead to a more efficient fluid ejection and aerosol formation and minimize the probability of chaotic membrane oscillations.

The inhalation device 100 may be attitude insensitive. More particularly, the placement of a surface tension plate behind and next to the aperture plate provides a constant and uniform fluid loading on the aperture plate and is not dependent on the overall orientation of the inhalation device 100 on fluid loading or placement or availability of fluid behind the aperture plate before actuation and spray. This may also provide a more efficient and stable aperture plate oscillation that may lead to a more efficient ejection of fluid and minimize the probability of chaotic membrane oscillations.

The inhalation device 100 may be rendered insensitive to pressure differentials that may occur when the patient 104 travels from sea level to sub-sea levels and at high altitudes while traveling in an airplane or in weather related changes in atmospheric pressure. In certain aspects, the application and use of a superhydrophobic air exchange valve placed adjacent to the drug reservoir in the cartridge 102 provides for protection against both water and drug intrusion or exchange across the valve and avoids leakage out of the reservoir. In certain implementations, the superhydrophobic air exchange valve, allows a free exchange of air across the valve into and out of the reservoir, while blocking moisture or fluids from passage through the air exchange valve. This high efficiency particulate air filter also normalizes the pressure differential between the environment and the reservoir, with highly filtered air, that removes all particles and pathogens, (including viruses pathogens, bacteria and spores), as the inhalation device 100 undergoes variations in atmospheric pressures and thereby experiences pressure differentials. The ability to provide for an active and dynamic mechanism for high efficiency air filtration also provides and prevents drug leakage, since by allowing free exchange of air the pressure inside the reservoir of the cartridge 102 is maintained in equilibrium with its environment.

The inhalation device 100 is insensitive to changes in ambient air pressure caused by variations in weather or altitude. This allows its use without the potential of fluid leakage as the inhalation device 100 transitions to different altitudes. The inhalation device 100 may also reduce fluid and drug deposition on aperture plate surfaces and prevent blockage of apertures which may prevent improper delivery of doses or render the inhalation device 100 inoperable.

The inhalation device 100 delivers a volume of fluid as an aerosol plume or fine mist of liquid riding on entrained air having a laminar flow as it travels into the pulmonary airways of the patient 104. One source of entrained air with the laminar flow is provided by a laminar flow element, with a plurality of parallel tubes having either hexagonal or other cross section, and placed at the air entrance end of the aerosol delivery tube of the inhalation device 100, as described herein. The laminar flow element generates a laminar air flow 106 by dividing the air flow into many parallel flow passages that have diameters that are sufficiently small to generate the laminar airflow 106 but not too small to create a significant pressure differential between the inlet side of the laminar flow element and exit side of the laminar flow element. Since only a small flow passes through each laminar flow element passageway, the Reynolds number of the flow through each passageway is small enough that laminar flow 106 is maintained. In certain implementations, the air drawn into the inhalation device 100 has the properties of a laminar flow, as air is drawn through the laminar flow element and sweeps across the face of the aperture plate, and entrains and carries the ejected particles in a trajectory further downstream into airways of the patient 104.

The inhalation device 100 may include a mini fan or centrifugal blower that is located at the air inlet side of the laminar flow element. The mini fan may provide additional airflow and pressure to the output of the airstream. If the patient 104 has a low pulmonary output, this additional airstream ensures that the drug containing droplets are pushed through the inhalation device 100 into the airway of the patient 104. In certain implementations, this additional source of airflow ensures that the ejector face of the cartridge 102 is swept clean of the aerosol droplets and also spreads the droplet plume into a stream which creates greater separation between droplets. The airflow provided by the mini fan may also act as a carrier gas, ensuring significant dilution, which reduces the likelihood of a sharp dose and rapid drug uptake. For certain medications, this rapid drug uptake could create unwanted side effects. In addition, a bladder squeezed manually or by an electromechanical component may be used to create a high air pressure during dosing to improve deposition of the medication in the lung for patient 104 where he has a low pulmonary output or is undergoing an exacerbation. A manual button on the inhalation device 100 may be used to trigger ejection of the medication during a “rescue” mode.

In one implementation, the inhalation device 100 provides a high modulus polymeric aperture plate whose periodic oscillation produces acoustic waves that emanate from the nozzle plate surface as it accelerates and decelerates during aerosol ejection cycles. The surface acoustic waves generate pressure waves or compression waves which further enhance generation and propagation of droplets on entrained air. The high modulus polymeric aperture plate with its plurality of openings ejects fluid as droplets through the entrance orifice and exit orifices contained in the accelerating aperture plate. In certain implementations, the oscillating high modulus aperture plate, when at its maximum displacement, the velocity at the surface of the aperture plate membrane is zero, while its acceleration has a negative maximum value in the range of 0.1-50 Km/s², 5-50 Km/s² but not limited to, 5-25 Km/s². The directed stream of aerosol droplets, directed through the openings in the thickness of the aperture plate surface, emerge from the openings with a maximum inertial force that is sufficient to carry the aerosol downstream, away from the aperture plate.

The inhalation device 100 may further employ Laser Doppler Vibrometer (LDV) systems and methods for characterization of electromechanical systems and piezoelectric actuators for measuring the instantaneous phase, displacement, velocity and acceleration of oscillating membranes. In certain aspects LDV may provide three dimensional representations of the phase relationships between displacement, velocity and acceleration to identify the resonances and Eigenmodes of electromechanical systems comprising of piezoelectric actuators that enable efficient optimization of piezoelectric materials, piezoelectric geometry optimization as well as design of ejector systems for optimum aerosol droplet generation and nebulization characteristics. Such analytics parameters may be detected, captured and stored simultaneously for subsequent analysis by the inhalation device 100 and/or an air analyzer.

The inhalation device 100 may deliver electrostatically charger pharmaceutical aerosol particles generated during ejection and droplet formation as the fluid emerges from the exit orifice of the oscillating nozzle plate. In one implementation, the surface of the exit chamber or tube through which the aerosol plume emerges is coated with a surfactant to reducing charge. The electrostatic coating may increase the fine particle dose delivered into pulmonary airways and reduce deposition of drug particles onto the inside surfaces of the device tube of the inhalation device 100. Alternatively, the exit tube or entire body of the inhalation device 100 may be made from a charge-dissipative polymer, which further reduces the electrostatic charge buildup in the exit chamber.

In one implementation, the inhalation device 100 includes a spray verification system for detecting pressure differentials between the interior and exterior areas of the ejector tube for verification of aerosol spray and drug delivery. A signal provided by pressure sensors of the spray verification system provides a trigger for activation of a spray at or during a peak period of an inhalation cycle of the patient 104 and assures optimum deposition of the aerosol spray and drug delivery into the pulmonary airways.

The spray verification system of the inhalation device 100 may include an infrared light emitting diode (LED), having a wavelength for example of approximately 850 nm, and an infrared photodetector. In one implementation, the spray verification system may use solar blind photo detectors and UV-C LEDs with peak emission wavelength below approximately 280 nm for measuring and sensing in either transmission or backscattering modes to detect the presence and quantity of ejected medication.

The spray verification system of the inhalation device 100 in a fluorescence mode where the air stream is exposed to an energy source such as ultra violet light and substances in the air stream fluoresce, emitting photons of light having a specific wavelength. This mode can be used to detect and measure a variety of airborne substances and provide spray verification with maximum detection and provide assurance of elimination of incorrect or faulty detection of spray. The solar blind detectors provide greater flexibility of use and operation of the inhalation device 100 with no interference when outdoors, in bright sunlight.

In another implementation, the inhalation device 100 uses an audio signal for spray verification when a dose is either dispensed by the breath actuation, and/or when an aerosol stream of droplets is detected. The addition of a sound chip to an electronics board of the inhalation device 100, with a speaker, provides immediate feedback to the patient 104 when a dose is successfully delivered. By providing real time feedback, the audio signal may maximize patient compliance by providing assurance that the dose was successfully delivered.

Alternatively or additionally, the inhalation device 100 may measure and quantify the amount of drug ejected during ejection and nebulization. Absorbance of a nebulized drug dose may be provided by measuring the absorbance of light. A drug solution may be previously calibrated using known concentrations to provide the drug absorbance values at specified wavelengths. The inhalation device 100 verifies that the drug was nebulized and ejected as well as provides the quantity and amount of drug in the ejected aerosol stream and the amount of drug remaining in the reservoir of the cartridge 102.

For diagnosis of pulmonary conditions and/or for air analytics, the inhalation device 100 may sense and detect disease biomarkers in exhaled breath. Human breath contains a number of volatile organic compounds (VOCs) whose accurate detection can provide essential information on the early diagnosis of diseases of the human or animal body in general. For example, nitric oxide can be used to evaluate or sense for asthma diagnosis; acetone, ammonia, H₂S, and toluene can be used to evaluate diabetes, kidney malfunction, halitosis, and lung cancer, respectively. Diagnosis of these diseases may be achieved by inclusion of miniaturized MEMS-based amperometric sensors. For example, inside the mouthpiece or droplet exit cavity of the device, a MEMS sensor may be used to analyze the concentration of nitric oxide in exhaled breath for ASTMA monitoring and diagnosis. Furthermore, variations in the concentration of the exhaled VOCs may serve as biomarkers for specific diseases and may be used to distinguish healthy patients from those who are sick.

The inhalation device 100 may include one or more visual, audio, and/or tactile indicators to provide instructions or feedback to the patient 104. The indictors may alternatively or additionally be provided via a user device in communication with the inhalation device 100, as described herein. For example, the inhalation device 100 may instruct the patient to initialize the inhalation device 100 by taking a set number (e.g., five) of slow deep breaths. This provides information used to determine a trigger point during inhalation to generate a breath actuated ejection of medication by the piezoelectric driven ejector to maximize the inhalation of the prescribed medication by the patient 104. It also provides a baseline for diagnosing changes in the pulmonary condition of the patient 104. For example, the minimum pressure achieved during inhalation corresponds directly to peak inspiratory flow. Decreases in peak flow during inhalation can be predictive of an asthma exacerbation. When peak flow measurements are combined with the measurement of exhaled nitric oxide, the predictive capability of the on board diagnostics configured on the inhalation device 100 and/or the air analyzer become even more powerful.

In one implementation, the inhalation device 100 captures measurements correlating to pulmonary output (FEV1) by measuring a maximum flow achieved during maximum exhalation. The air analyzer, described herein, provides prompts to the patient 104 using the indicators to lead the patient 104 through the breathing exercises required to make measurements that correlate to pulmonary output.

In addition, the inhalation device 100 may contain differential (delta P) sensors to allow measurement of the inhalation pressure required to achieve a predetermined flow. This measurement may allow the device to approximate changes in dynamic airway resistance by comparing the pressure required to create the flow of air measured during initialization. While not a measurement of absolute airway resistance, this measurement of the change in the pressure measured at one side of the airway at a given flow during inhalation measures changes in the pulmonary health of patient 104, where he suffers from asthma or COPD. This may be referred to herein as a relative change in dynamic airway resistance (RCDAR), which is expressed as the percentage change in the pressure measured at 50% of the flow of air measured at baseline. For example, a reading of 10% RCDAR is a 10% decrease in the pressure required to achieve 50% of the maximum flow measured at baseline. Additionally, the inhalation device 100 can be used to confirm the velocity of the ejected plume before inhalation and may act as a confirmation that the flow sensors of the inhalation device 100 are correctly calibrated and operating.

The delta P sensor measures the airflow by measuring the pressure drop between the interior of the inhalation device 100 and the surrounding atmosphere. Flow rate is calculated from the measured pressure drop between the delta P sensor ports; one located upstream in the device aerosol delivery tube, near the air inlet port, in the vicinity of the laminar flow element, while the second delta P sensor port measures ambient pressure outside the inhalation device 100, as described in more detail herein. This measurement is also used to trigger the beginning and ending of an ejection cycle of droplets in order to coordinate the optimum point of the inhalation cycle with ejection and spray of the aerosol plume. The pressure measurement subsystem also differentiates between inhalation and exhalation so that droplet particles are only dispensed on inhalation during the inspiratory cycle.

In one implementation, optical aerosol sensors of the inhalation device 100 measure and detect the presence of droplets by detecting light emitted from an LED source placed across the diameter of the inhalation tube and detecting the light scattered or absorbed by the droplets by a photodetector. The light source is a narrow viewing angle (e.g., less than 8 degrees) LED or a laser diode. In addition, multiple light sources and multiple detectors placed, for example, along the inhalation device 100 exit port or in front of the ejector plate of the cartridge 102, may determine a shape, including a cross-section and length, of the aerosol plume for estimating the ejected mass.

For example, where the inhalation device 100 includes a flow tube having an average diameter of 20 mm, a four-second inhalation of air from 100 milliliters to 500 mL will have an average velocity of from 8 to 40 centimeters per second. With the optical sensor located 20 mm downstream from the ejector assembly of the cartridge 102, the front edge of the aerosol particles will arrive at the optical detector from 50 to 250 milliseconds after ejection.

Typical photodetectors which may be used in the inhalation device 100 may have response times of less than 1 millisecond, thus allowing accurate resolution of entrained droplet velocity. A second LED/photodetector system may be added and used to provide finer resolution of aerosol velocity. In one implementation, the inhalation device 100 measures and detects the arrival of the aerosol plume at two downstream points, several centimeters apart. In this case, the LED source for each system is pulsed and synchronous detection is used so synchronize each detector with its associated light source.

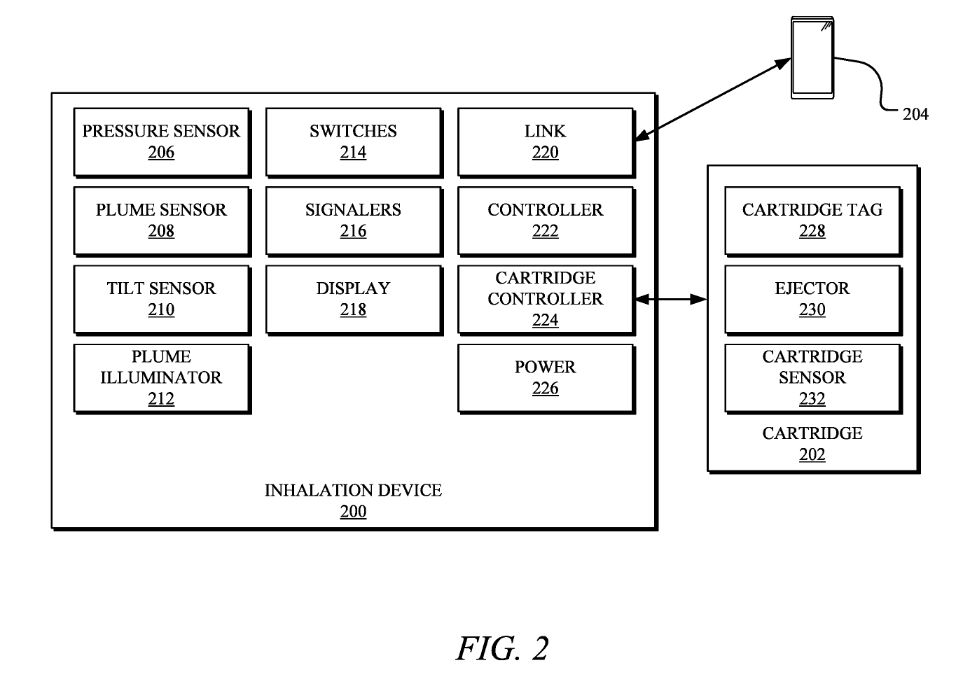

To continue a detailed description of a particular implementation 200 of the inhalation device 100, reference is made to FIG. 2. The various components of the inhalation device 200 coordinate all operational functions, as well as archive and communicate captured air pulmonary health management data, including spray validation data, diagnostic indicators, and device operational parameters, for generating air analytics. In addition, the identification of a cartridge 202 or dose size parameters may also be stored in memory the inhalation device 200 and/or the cartridge 202. For onboard data storage, a controller 222 may include a low-power microprocessor having several kilobytes of data memory, several channels of 10 to 12 bit analog to digital conversion, and several digital outputs. However, other controllers are contemplated.

The inhalation device 200 may include a pressure sensor 206, a plume sensor 208, a tile sensor 210, a plume illuminator 212, switches 214, signalers 216, display 218, a wired or wireless link 220 to one or more user devices 204, a cartridge controller 224, and a power supply 226. The cartridge controller 224 may control operations of and/or capture information from the cartridge 202, including a cartridge tag 228, an ejector assembly 230, and a cartridge sensor 232. It will be appreciated that more or fewer of these components may be included in the inhalation device 200.

In one implementation, the controller 222: activates when a power button is pressed; monitors the pressure sensor 206; waits for a dispense command or inhalation profile of a rapidly descending pressure; activates the cartridge controller 224, such as a piezo driver with a predetermined frequency and voltage; monitors the cartridge controller 224 to assess dispense conditions; monitors a flow rate and plume using the pressure sensor 206 and the plume sensor 208, respectively, to dispense at the trigger point; and appropriately illuminates signalers 216, such LEDs, on and off to signal an effective or ineffective dispense or provide other user feedback.

The controller 222 may have an internal clock to wake up and signal when the next dose for the patient 104 is scheduled. This event may be signaled by the signalers 216, such as a blinking LED or a piezo buzzer. Alternatively, an alert may be output to the user device 204 via the link 220. The controller 222 is configured to operate with or without communication with the user device 204 and accordingly stores the time and success of each dispense. The profiles of the inhalation flow and plume in with 0.1 to 0.01 second resolution for the five to ten second interval surrounding a dispense may further be stored. Additionally, if the inhalation device 200 has the tilt sensor 210, such as an attitude or piezo mesh sensor, that data may also be stored or processed in real time to indicate a good or potentially good dispense. The controller 222 may also track and display the number of doses and remaining doses left in the cartridge 202 using the display 218 and/or the user device 204.

The power supply 226 may include one more power sources configured to store and provide power, such as solar power, battery power, or DC or AC power, to the components of the inhalation device 200. In one implementation, the power supply 226 includes a non-rechargeable battery system such as lithium polymer. There may be a connector on the inhalation device 200 to charge such a battery or other methods of charging including but not limited to solar cells, inductive links or even mechanical motion. The controller 222 may be configured to deactivate at very low levels of power consumption and still monitor input buttons and respond to a press by going to the fully awake state.

In one implementation, in order to insure and maximize patient compliance and to insure proper use of the aerosol delivery device, the inhalation device 200 and/or the user device 204 may convey a variety of information to the patient 104. Examples such information to promote adherence to the prescribed aerosol therapy includes, without limitation: device ON, good/bad dispense, number of remaining doses, time for another dose, and explanation of an error condition for the inhalation device 200 (like incorrect orientation for delivery, or no plume seen). Some of this information may be communicated with two to three light sources of the signalers 216: one to signal the unit is on and the other to signal successful or unsuccessful dispense. Two or three color (red-green-yellow) LEDs can be used for more user-friendly communication of information. Error codes based on blinks of either or both LEDs are possible. Similarly a four digit LCD display can be used to show the number of remaining doses or an error code. Explanation of error codes or more detailed information can be communicated via the user device 204. One or two user input buttons turn on (wake up) the inhalation device 200 and can initiate a dispense or request an error code or provide remaining dose information.

The link 220 may facilitate archiving, analysis and training for the patient 104 and can communicate use and lung/airway diagnostics to a health provider via the user device 204. The user device 204 can also be a resource for understanding correct use and diagnosis of the inhalation device 200. The user device 204 can also convey password-protected information back to the inhalation device 200 from the health provider such as a revised dose quantity or frequency based upon breathing flow rates measured by the device with or without an actual dispense. In one implementation, the user device 204 can initiate a reorder when remaining doses go below a preset limit.

The link 220 can also be used at a clinical facility or pharmacy to set basic parameters or retrieve archived information from the inhalation device 200. This information includes the following setups that may be communicated to the device, without limitation: dispense duration, ejector frequency, number of breaths per dosing, inhalation flow that triggers dispense, desired dose interval (e.g., twice a day or once every twelve hours), max number of good doses allowed in a time period (for example, two hours), max number of error doses allowed in a time period, number of doses dispensed until an almost empty signal given, parameters that describe good dose (inhale volume post dispense, plume opacity), date/time, expiration date or interval for the drug, password for use with provider office or pharmacy office to change settings, and/or other information describing good or bad dose limits.

In one implementation, the piezoelectric ejector frequency and voltage of the ejector assembly 230 may be preset during factory filling of the cartridge 202. Such values may be modified using the user device 204 via the link 220. Similarly, fill or expiration dates, drug type or even a security code to guard against generic use may be carried by the cartridge tag 228. Examples of information that might be stored and/or communicated via the cartridge tag 228 may include, without limitation: dose volume and dose interval (times per day); acceptable inhalation flow rates or characteristics; and/or options for allowing repeated doses if a bad dosage is sensed.

There are three additional components that may be part of the inhalation device 200, such as mesh monitoring, orientation monitoring (e.g., via the tilt sensor 210) and reservoir fluid measurement components. Mesh monitoring allows immediate determination that the piezo mesh is vibrating correctly by sensing the vibration with a small piece of piezo material mechanically coupled to the mesh. The electrical output from the small piezo can be amplified, rectified and measured using one analog-to-digital input of the controller 222. This can supplement or replace mesh monitoring done by the electronic driver impedance measurement. Orientation monitoring determines that the inhalation device 200 is oriented correctly so that fluid in the reservoir wets the back of the mesh for good droplet generation. This same attitude measurement sensor can also be used to verify the amount of fluid (hence, doses) remaining in the reservoir. This is accomplished by slowly tilting the inhalation device 200 until fluid is sensed either by a conductive wire, a capacitive sensor on the reservoir housing or an optical sensor of the fluid. The tilt is noted at the instant the fluid sensor indicates a transition from sensing no fluid to sensing the fluid. A simple algorithm (or lookup table) in the controller 222 gives the amount of fluid remaining as a function of the tilt angle. This function can be used for a more exact assessment of the number of doses remaining in the cartridge 202, and is especially important when a pharmacy blindly fills the reservoir and a confirmation of the remaining doses available is needed.

As described herein, the inhalation device 200 generates droplets and controls operation and monitoring of both the inhalation flow and plume to verify an efficacious delivery of the drug. The following are examples of how the patient 104 operates the inhalation device 200 in different user modes. In a manual dispense mode, the patient 104 turns on and presses dispense button, the inhalation device 200 signals plume seen at correct inhale window, and the patient 104 turns off the inhalation device 200 or it automatically deactivates. In a flow triggered dispense mode, the patient 104 activates the device and inhales. The inhalation device 200 triggers a dose at a trigger point corresponding to a specific inhale flow rate, set by a provider. The inhalation device 200 signals good dispense and powers down. In an trained triggered dispense mode, the patient 104 turns on the inhalation device 200 and breaths several times for full breathing cycles of inhalation and exhalation. The inhalation device 200 records and dispenses the dose at the trigger point, which is automatically detected using the pressure sensor 206. The inhalation device 200 signals a good dispense and deactivates. In a training or diagnostic mode, the patient 104 turns on the inhalation device 200, and the inhalation device 200 or the user device 204 prompts the patient 104 through breathing cycles. Diagnostic indicators and other pulmonary health data is captured with the inhalation device 200, which is then archived on the controller 222 and/or communicated via the link 220 and/or user device 204 to an air analyzer. The inhalation device 200 deactivates.

In one implementation, of a normal automatic mode of the inhalation device 200, the patient 104 is prompted to deliver a dose. The inhalation device 200 and/or the user device 204 instructs the patient 104 to breathe one or more cycles and press the dispense button. The inhalation device 200 detects an inhale and ejects plume into airstream. If the pressure sensor 206 shows correct breath and dispense timing then signalers 216 provide feedback, such as a light blinking green. If the dose is poorly administered, then a red or yellow light of the signalers 216 blinks. If yellow light of the signalers 216 blinks, then the patient 104 is prompted for another dispense and correct or incorrect dispense is signaled. After 30 seconds of no activity the controller 222 turns off the inhalation device 200. If there are a plurality of poor dispenses, an error code is generated by the controller 222 and communicated to the patient 104, for example using the user device 204. The signalers 216 may include a visual, audio, and/or tactile signal prompting the next dose. A counter may be displayed via the display 218 including a number of doses remaining.

The user device 204 and/or inhalation device 200 may provide feedback to the patient 104 and/or communicate information to the provider, as described herein. The feedback may include information regarding any errors, including potential remedies and error codes, which may include, without limitation, no cartridge, wrong drug cartridge, no plume seen, out of doses according to count, insufficient inhalation, low battery, ejector vibration problem, dose request incompatible with prescription or gather diagnostic information, and/or the like.

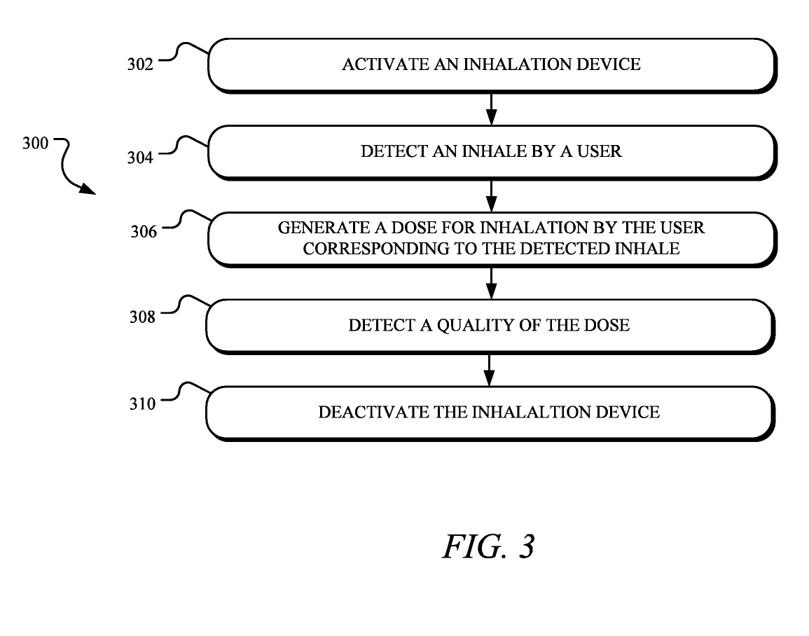

Turning to FIG. 3, operations 300 for medication delivery for a patient using an inhalation device are shown. In one implementation, an operation 302 activates an inhalation device, and an operation 304 detects an inhale by a user. An operation 306 generates a dose for inhalation by the user corresponding to the detected inhale. An operation 308 detects a quality of the dose, and an operation 310 deactivates the inhalation device.

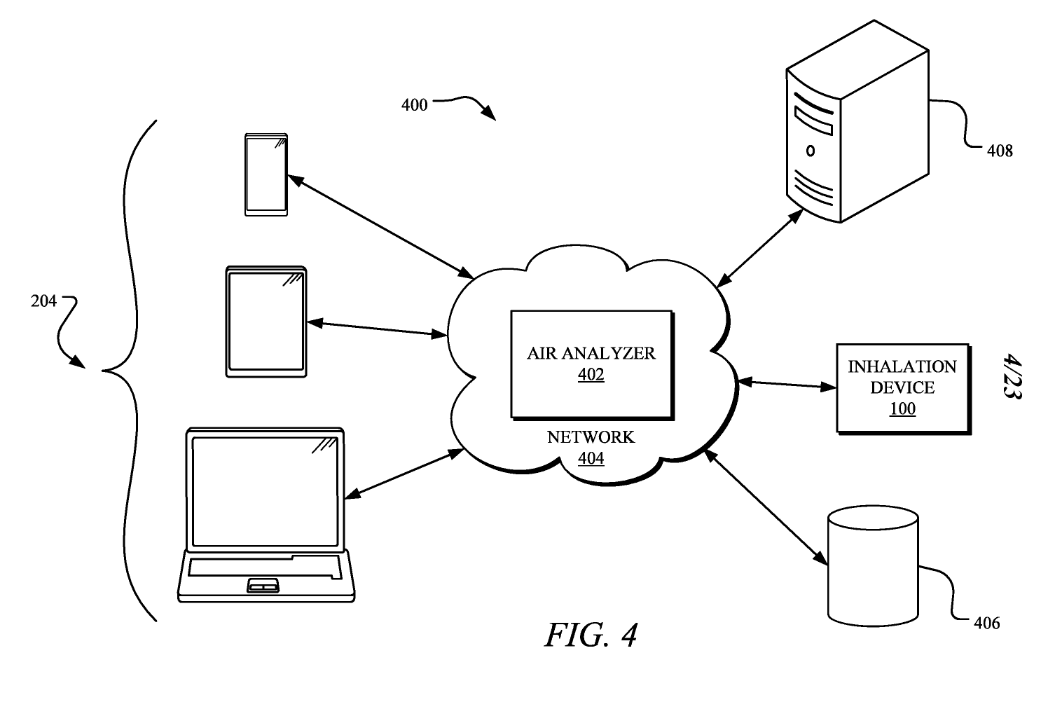

FIG. 4 is an example pulmonary health management system 400, including an air analyzer 402 running on a computer server, computing device, or other network device, for pulmonry health management. In one implementation, a user, such as the patient 104, accesses and interacts with the air analyzer 402 and/or one or more inhalation devices 100 via a network 406 (e.g., the Internet). In another implementation, the user device 204 locally runs the air analyzer 402, and the inhalation device 100 connects to the user device 204 using a wired or wireless connection. Other users may be one or more parties that sell, operate, manage, and/or otherwise monitor the inhalation devices 100 including a physician, health clinic, health laboratory, and/or the like.

The network 404 is used by one or more computing or data storage devices (e.g., one or more databases 406) for implementing the pulmonary health management system 400. The user may access and interact with the air analyzer 402 using the user device 204 communicatively connected to the network 404. The user device 204 is generally any form of computing device capable of interacting with the network 404, such as a desktop computer, workstation, terminal, portable computer, mobile device, smartphone, tablet, multimedia console, and/or the like. The user device 204 may be a patient user device, a provider user device, and/or the like depending on the user.

A server 408 may host the pulmonary health management system 400. The server 408 may also host a website or an application, such as the air analyzer 402 that the user visits to access the system 400. The server 408 may be one single server, a plurality of servers with each such server being a physical server or a virtual machine, or a collection of both physical servers and virtual machines. In another implementation, a cloud hosts one or more components of the system 400. The one or more inhalation devices 100, the user devices 204 employed by the users, the server 408, and other resources, such as the one or more databases 406, connected to the network 404 may access one or more other servers for access to one or more websites, applications, web services interfaces, etc. that are used for pulmonary health management. The server 408 may also host a search engine that the system 100 uses for accessing and modifying information used for pulmonary health management.

The inhalation devices 100 communicate with the air analyzer 402 executed by the user device 204 via a wireless connection, such as Bluetooth, over the network 404, or via a wired connection, such as a USB connection. The inhalation devices 100 may communicate in similar manners with other computing devices, such as a smart watch, smartphone, tablet, computer, music player, Bluetooth enabled devices, and the like.

In one implementation, the inhalation devices include one or more sensors for capturing pulmonary health data, including diagnostic indicators, operational parameters of the inhalation devices 100, spray verification data, and/or the like. The sensors may include, without limitation, one or more pressure sensors, plume sensors, tilt sensors, humidity sensors, temperature sensors, particle sensors, heart rate sensors, carbon dioxide sensors, oxide sensors, ozone sensors, nitric oxide sensors, microphones, imaging sensors, and/or the like. Such data may be stored in storage media of the inhalation devices 100 and/or communicated to the air analyzer 402 via the user device 204. By way of example, the data captured by the sensors may be retrieved and stored on the consumer device 204 and/or uploaded to a secure cloud over the network 404 to the databases 406.

Once the data is obtained by the air analyzer 402, it can be utilized in many ways by the patient 104, the provider, and other approved parties. As explained herein, inhalation devices 100 generate large amounts of sensor data that the air analyzer 402 may correlate to generate associations and understanding patterns and trends to improve healthcare, save lives and reduce cost. In addition, air analytics generated by the air analyzer 402 may provide clinical decision support, disease state surveillance and healthcare management, in real time, while the patient 104 is undergoing treatment.

The air analyzer 402 generates air analytics that include a date/time for each dispense as well as inhalation flow rates, which not only provides key individual information for the provider but also provides valuable data for collective analysis that supports the patient 104, as well as other group of sufferers of pulmonary diseases, such as asthma and COPD. The air analytics generated by the air analyzer 402 may alert or warn an individual as well as a group of users as to a risky or challenging health exacerbation or environmental condition. The air analytics for a particular patient may contain, for example, comparisons of pattern of use of the inhalation device 100, inspiratory flow rates for the patient 104, frequency of use, medication, and dosing information. Other diagnostic indicators may be recorded and stored in the database 406, such as airway resistance, inspiratory volumes, inspiratory vital capacity and corresponding diagnostics, Peak Inspirational Flow (PIF) and Maximum Inspirational Flow at 50%, (MIF at 50%).

The air analyzer 402 may generate the air analytics through a comparison of pulmonary health management data with previous stored data to establish when there is a change in use of the inhalation device 100 or changes in pulmonary function for the patient 104 and may further predicting an acute exacerbation of COPD. Pulmonary health management information from a plurality of patients may be anonymized by the air analyzer 402 to determine general use patterns, inspiratory volume and vital cycle conditions, date/time and regional location of each use, general user classification data, such as disease type and severity, age, drug type(s), and generalized patient history. Operational parameters for each of the inhalation devices 100 may further be captured and processed by the air analyzer 402 for use in air analytics generation for troubleshooting, improvements, and/or the like.

The air analytics may be used predict and determine regional locations and times of the day that pose the greatest threat to patients with similar conditions. For example, if a decreased inspiratory vital capacity or increased use of the inhalation devices 100 is reported for asthma sufferers in a specific area, perhaps due to an unidentified environmental condition, such as pollen or other allergen, then a warning may be communicated via the user device 204 and/or inhalation device 100 to others in that area with asthma or other pulmonary conditions as a warning to suggest that they stay indoors. Such a system may also factor in and incorporate regional or local environmental monitoring by the government or a third party.

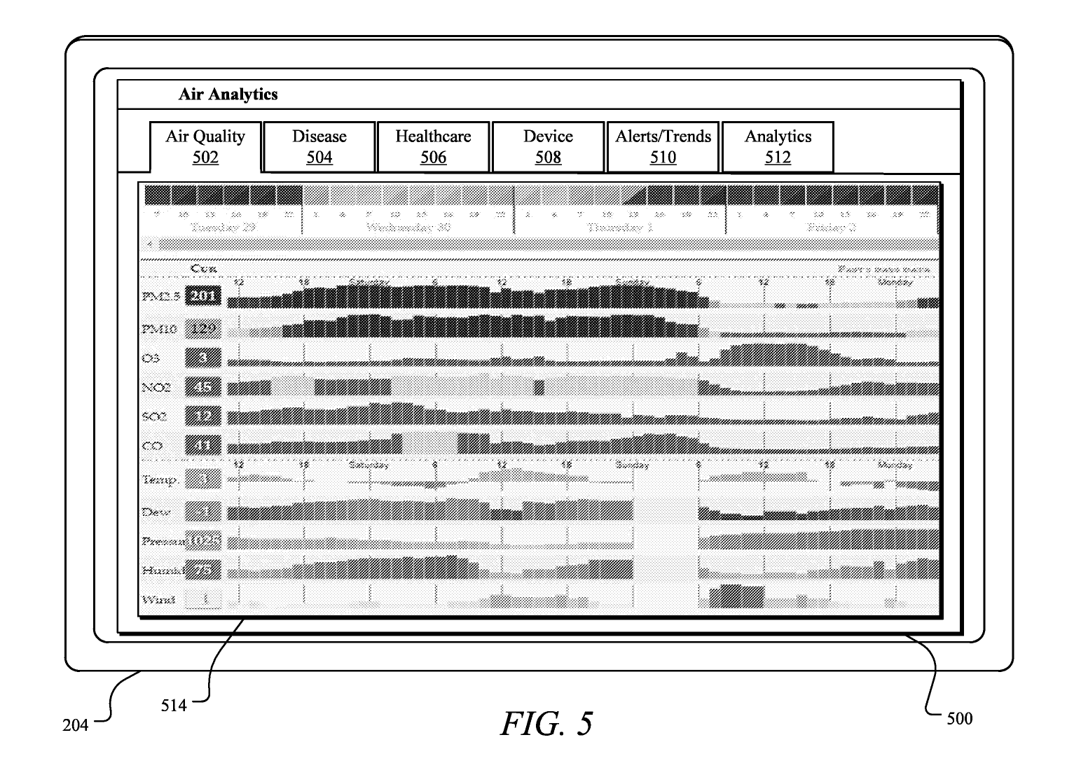

Referring to FIG. 5, an example air analytics user interface 500 is shown. The air analytics user interface 500 is generated by the air analyzer 402 and displays air analytics, including, without limitation, air quality 502, disease information 504, healthcare information 506, device information 508, alerts/trend 510, and other analytics 512. The navigation tabs 502-512 are exemplary only and not intended to be limiting.

The air quality 502 may include air analytics for a particular geographical location(s) based on pulmonary health management information and environmental data captured using one or more environmental sensors deployed in the ambient atmosphere of the geographical location(s). The air quality 502 may include an air quality interface 514 providing air quality monitoring and air pollution for geographical location(s), such as Beijing, China. The air quality interface 514 may include correlations and effects of various air pollution conditions and contaminants on respiratory diseases. For example, air quality factors such as PM2.5, PM10, O₃, NO₂, SO₂, and CO, in addition to weather conditions such as temperature, dew, pressure, humidity and wind speed are employed as inputs captured by the environmental sensors to monitor or predict the onset of respiratory problems or to generate warning to suggest to stay indoors.

The disease information 504 may include diagnostic indicators captured by the inhalation devices 100 correlated for one or more patients to diagnose pulmonary conditions. A summary of the incidence of disease conditions may be provided for particular geographical location(s) to identify any environmental or other causes, as well as to monitor an overall disease state of the population. The healthcare information 506 may be used by an individual patient or group of patients to track treatment, diagnosis, disease state, and/or the like. The device information 508 may include information on the operation of the inhalation devices 100 for troubleshooting, improvements, quality control, and/or the like. Various alerts and/or trends corresponding to the various tabs 502-508 and 512 may be included in the alerts/trends 210 for management of one or more patients, groups, and/or the inhalation devices 100.

For example, correlation of real-time reported occurrences of acute exacerbations of COPD with recorded environmental conditions may lead to refined warning levels as well as provide a better understanding and provide potential discovery of trends and patterns of the effects of environmental irritants, in combination with prescribed medication effects and dosage levels, on patient recovery and outcome. The use of air analytics and real-time sensing and reporting quickly identifies improved dosing regimens or drug combinations or provide a means for quickly identifying problematic or potentially illegally tampered batches of medication. The air analytics are valuable for medical and pharmaceutical research, for clinical decision making and disease surveillance which may lead to improved healthcare, save lives and lower costs.

As described above, the inhalation devices 100 collect and distribute information beyond inhaler use and inhalation parameters. Additional user data may include inhalation air temperature, humidity, ozone and nitrogen dioxide levels, amount of various gases in the patient 104 exhalation, and even particulate levels in the inhaled air. Conveyed to the air analyzer 402, warnings may be issued to an individual user or user group, based on real-time environmental and pulmonary health management information.

The air analyzer 402 provides personalized healthcare by providing a means for comparing and optimizing the effectiveness of individual therapies with outcomes of larger groups. The air analyzer 402 discovers relationships between prescribed therapy, air pollutants or other environmental conditions. This information may be used for improving healthcare or predict impending exacerbations for the user. The combination of individual measurement and real-time support from the air analyzer 402 improves the quality of healthcare delivery, while reducing the cost as well as provide insights for making better informed healthcare decisions.

Air analytics generated by the air analyzer 402, as applied to personalized healthcare, warns the patient 104 based on their own pattern of use of their inhalation device 100 when visiting different environments. For example, a patient who occasionally visits the home of a friend where formaldehyde or other noxious vapors are emitted by a rug or building material, may reveal their effects later on in the day and lead to changes their inhalation device 100 use and dosing pattern. The air analyzer 402 detects this change in pattern and informs the patient 104 of the development of a new trend of a more frequent dosing regimen after spending time at a specific location and warn the user of the newfound correlation.

The air analyzer 402 may apply air analytics to the inhalation devices 100 to sense and detect disease biomarkers in exhaled breath. Human breath contains a number of volatile organic compounds (VOCs) whose accurate detection can provide essential information on the early diagnosis of diseases of the human or animal body in general. For example, nitric oxide can be used to evaluate or sense for asthma diagnosis; acetone, ammonia, H₂S, and toluene can be used to evaluate diabetes, kidney malfunction, halitosis, and lung cancer, respectively.

A relative measurement of lung function made during dosing or at another time can furnish valuable information to the user or doctor. In conjunction with the air analyzer 402, it may be used to teach the user when or how to administer a dose of drug with the help of the healthcare provider. Lung function measurement can be used to teach the user when to take action and to provide him relative lung function information. This education may be done via the air analyzer 402, which shows the user the extent that lung function has declined in various situations.

One useful diagnostic indicator for airway obstructive diseases is called FEV1, which is the forced expiratory volume of air in 1 second. Ideally a lung function measurement would be made so that it is independent of the users muscle effort to breath, however, current medical diagnostic equipment eliminates the variable factor of muscle effort to breath by use of a chamber surrounding the user which varies the pressure surrounding the user to evaluate the airway and alveolar restrictions on breathing. Typically FEV1 is expressed as the volume of air exhaled in one second by a forced expiration following a full inspiration. This number is expressed as a percentage of the vital capacity.

The availability of FEV1 values measured multiple times a day over multiple days can provide the doctor valuable diagnostic indicators for air analytics. In restrictive lung diseases (such as pulmonary fibrosis), the vital capacity is reduced to below normal levels, however the rate of exhalation of is normal. In obstructive lung diseases (such as asthma, emphysema, bronchitis) the vital capacity is normal because the lung tissue is not damaged and its compliance is unchanged. In asthma the small airways (bronchioles) constrict, increasing the resistance to airflow. Although the vital capacity is normal, the increased airway resistance makes expiration more difficult and takes longer time. Obstructive disorders are diagnosed by tests that measure the rate of forced expiration such as the FEV1 and FEV25-75, significant decrease in these values suggest an obstructive lung disease.

A lung diagnostic system provided by the air analyzer 402 can be accomplished with the inhalation device 100 by measuring flow rate over several breaths by the user with the same sensor used to time dispense to an optimum period during inhalation. Diagnostic indicators may be derived by comparison of each test to a benchmark pattern or number performed under the supervision of the doctor of the patient 104. Comparisons may be performed with a portion of the inhalation cycle or just the peak measured flow in each inhalation or exhalation.

To measure FV1 the user is first asked to do several forced expirations after full inhalations while the inhalation device 100 measures and records the flow rates. FEV1 is then calculated by integrating the flowrates over the entire exhale as well as the one-second interval. These results are stored and later or immediately communicated to the air analyzer 402. If the calculated FEV1 is below a preset limit then the device provides feedback that airway obstruction is greater than the preset value. The preset value can be determined by a medical professional and provided via the air analyzer 402.

The diagnostic indicators listed above is not a complete list of all possible diagnostics. For example, alveolar restrictions might be inferred from the relative differences in inhalation and exhalation flow rates with a decrease in the inhalation rate relative to the exhalation rate inferring more restriction in expanding the alveoli. Integration of flow rate over the entire inhalation and/or exhalation portions of one or more breathing cycles estimates the total amount of air inhaled. Like FEV1, such a value might be measured and recorded multiple times a day.

In addition to peak and one-second flows, the inhalation device 100 can do more sophisticated methods of assessing airway resistance and even lung compliance. For example, the effect of airway resistance is modeled with the air analyzer 402 by the equation: F=(P1−P2)/R, where airflow, F (frequently called dV/dt), is equal to pressure in the mouth, P1, minus alveolar pressure, P2, divided by airwave resistance, R. P2 is difficult to directly measure since this is the pressure in the lung's alveolar sacks. However, by measuring the flow rates at two different values of P1 (pressure in the mouth) for a relatively constant alveolar pressure, the airway resistance R can be calculated as follows:

For mouth pressure P1a the flowrate, Fa=(P2−P1)/R

For mouth pressure P1b the flowrate Fb=(P2−P1b)/R

This flows and pressures at these conditions can be used to directly calculate a value for airway resistance by:

R=(P1b−P1a)/(Fa−Fb)

Note this assumes relatively quick transitions between the two mouth pressures since both airway resistance and alveolar pressure change significantly with the changes in lung and airway geometry during a breathing cycle. By a quick transition of pressures the assumption of nearly constant alveolar pressure during the two pressures is true. There are limits to how quickly the pressures can be varied since the compliance of the air and flow resistance effectively eliminate the variation much as a low-pass filter minimizes high frequency signals.

The inhalation device 100 may generate two different mouth pressures required by the formula above by various means, such as a rapidly changing pressure source like a cylinder and piston, or even a long-throw audio speaker. Both these ways and means require significant volume, weight and power. Similarly, a direct or centrifugal fan may be used. However, the inertial force generated within these devices may make it difficult to measure the rapid changes in pressure that occur over a single breath.

In one implementation, the air analyzer 402 uses a variable resistance valve between the mouth and atmosphere to achieve the small and rapid pressure changes incurred during a respiratory cycle. By way of a non-limiting example, a flapper valve may be used to open and close part of the airflow path from atmosphere into the inhaler. This value may consist of a rotating disk, where half the disk is open and half is nearly blocked, so that as it spins it fully opens or partially closes the air inlet port to the inhaler. Such a disk can easily be rotated at 1 to 10 Hz with a small, relatively low-power 60 to 600 rpm miniature gear motor. When the disk is at the open position, C, with flow resistance, Rc, the flow rate into the mouth is: Fc=Pc/Rc.

Here Pc is the pressure in the mouth measured relative to the atmosphere. When the disk is at the restricted flow condition, D, the flowrate into the mouth, Fd=Pd/Rd. Substituting these two flow and pressure conditions in the above equation yields that airway resistance,

R=(P1c−P1d)/(P1c/Rc−P1d/Rd).

For the case where the inhaler flow restriction from condition C to D is increased by a factor of K=Rd/Rc, then the equation for airway resistance simplifies to:

R=(P1c−P1d)/(P1c/Rc−P1d/KRc) or R=(1/Rc)*(P1c−P1d)/(P1c−P1d/K).

Thus, by rapidly changing the flow resistance in the inhalation device 100, the airway flow resistance can be directly estimated by the air analyzer 402.

The air analyzer 402 may generate additional air analytics about lung compliance and the position of airway resistance can be evaluated by doing the above two-pressure test with different cycling times. In this case, instead of cycling between two different pressures the flow resistance in the inhalation device 100 is varied as a sinusoid to approximate the assumptions of linear signal processing.

In the case that a patient has significant airway resistance and cannot tolerate extra airflow resistance from the inhalation device, a small fan can be used to create additional pressure at atmospheric side of the variable resistance valve. Additional pressure can be generated by stacking the fans so that the total pressure is the sum of the pressure gains in each fan.

The air analyzer 402 measures airway resistance for in patient diagnosis by comparing a current airway resistance of the patient 104 to previous data generated by the device inhalation 100. Comparative data can also be used to evaluate the effectiveness of the drug droplets inhaled to ameliorate a restricted airway. The inhalation device and/or the air analyzer 402 may further track a varying pressure source or flow restriction, monitoring of internal pressure before and after administration of various drugs.

Because of the ability of the inhalation device 100 and/or the user device 204 to store, process and communicate data, the inhalation device 100 may contain additional sensors that relate to diagnostic or warnings to the user. For example, an N02 sensor might be added to the device to provide additional diagnostics for airway condition and flow resistance and to help both the user and his doctor understand how the patient 104 is responding to an asthma situation.

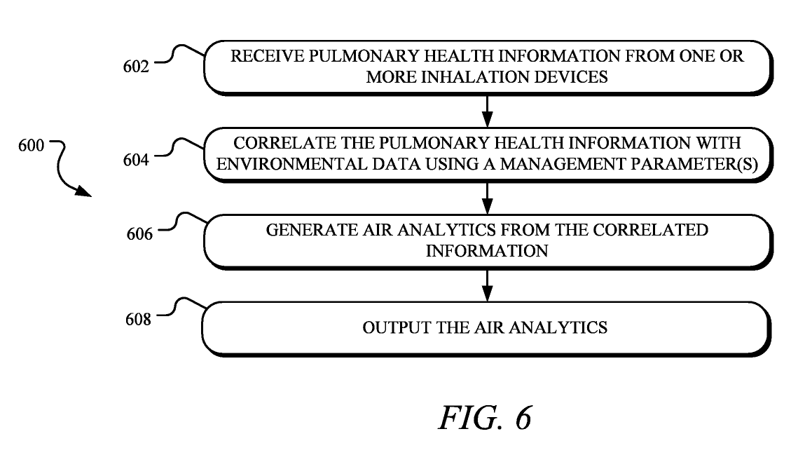

FIG. 6 illustrates example operations 600 for pulmonary health management for one or more patients. In one implementation, an operation 602 receives pulmonary health data from one or more inhalation devices over a network. An operation 604 correlates the pulmonary health data with environmental data using at least one management parameter. An operation 606 generates air analytics from the correlated data, and an operation 608 outputs the air filtration analytics.

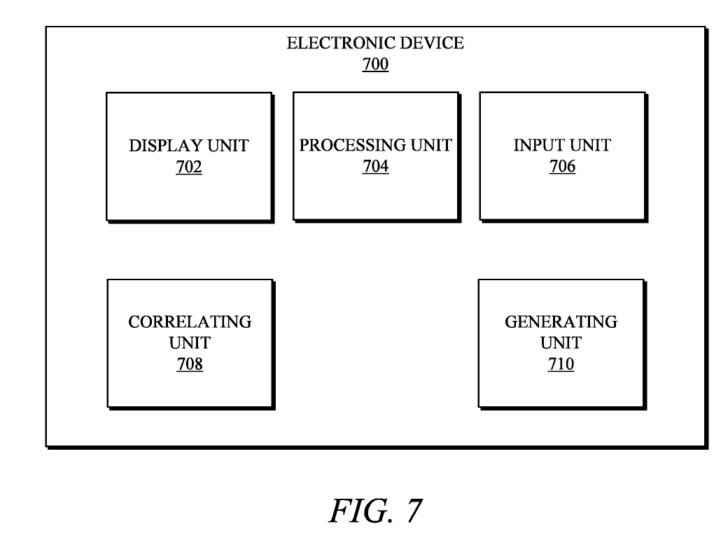

Turning to FIG. 7, an electronic device 700 including operational units 702-710 arranged to perform various operations of the presently disclosed technology is shown. The operational units 702-710 of the device 700 are implemented by hardware or a combination of hardware and software to carry out the principles of the present disclosure. It will be understood by persons of skill in the art that the operational units 702-710 described in FIG. 7 may be combined or separated into sub-blocks to implement the principles of the present disclosure. Therefore, the description herein supports any possible combination or separation or further definition of the operational units 702-710.

In one implementation, the electronic device 1700 includes a display unit 702 to display information, such as a graphical user interface, and a processing unit 704 in communication with the display unit 1702 and an input unit 706 to receive data from one or more input devices or systems, such as the air analyzer 402, the inhalation devices 100, and/or the like. Various operations described herein may be implemented by the processing unit 704 using data received by the input unit 706 to output information for display using the display unit 702.

Additionally, in one implementation, the electronic device 700 includes an correlating unit 708, and an air analytics generating unit 710. The correlating unit 708 correlates pulmonary health information with environmental information and the air analytics generating unit 710 generates air analytics using the correlated information.

In another implementation, the electronic device 700 includes units implementing the operations described with respect to FIG. 6. For example, the operation 602 may be implemented by the input unit 706, the operation 604 may be implemented by the correlating unit 1108, the operation 606 may be implemented by the fair analytics generating unit 710, and the operation 608 may be implemented by the output unit 702.

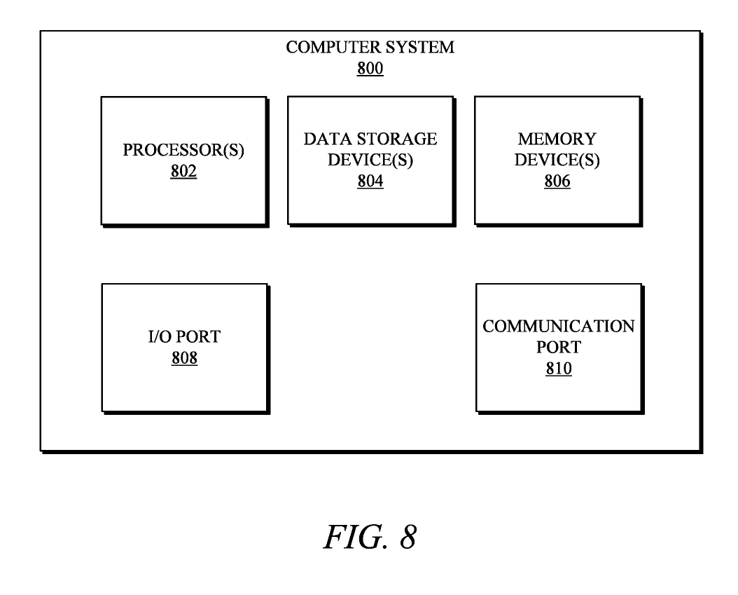

Referring to FIG. 8, a detailed description of an example computing system 800 having one or more computing units that may implement various systems and methods discussed herein is provided. The computing system 800 may be applicable to the air analyzer 402, the controller 222, the server 408, the inhalation device 100, and other computing or network devices. It will be appreciated that specific implementations of these devices may be of differing possible specific computing architectures not all of which are specifically discussed herein but will be understood by those of ordinary skill in the art.

The computer system 800 may be a computing system is capable of executing a computer program product to execute a computer process. Data and program files may be input to the computer system 800, which reads the files and executes the programs therein. Some of the elements of the computer system 800 are shown in FIG. 8, including one or more hardware processors 802, one or more data storage devices 804, one or more memory devices 808, and/or one or more ports 1808-1810. Additionally, other elements that will be recognized by those skilled in the art may be included in the computing system 800 but are not explicitly depicted in FIG. 8 or discussed further herein. Various elements of the computer system 800 may communicate with one another by way of one or more communication buses, point-to-point communication paths, or other communication means not explicitly depicted in FIG. 8.

The processor 802 may include, for example, a central processing unit (CPU), a microprocessor, a microcontroller, a digital signal processor (DSP), and/or one or more internal levels of cache. There may be one or more processors 802, such that the processor 1202 comprises a single central-processing unit, or a plurality of processing units capable of executing instructions and performing operations in parallel with each other, commonly referred to as a parallel processing environment.

The computer system 800 may be a conventional computer, a distributed computer, or any other type of computer, such as one or more external computers made available via a cloud computing architecture. The presently described technology is optionally implemented in software stored on the data stored device(s) 804, stored on the memory device(s) 806, and/or communicated via one or more of the ports 808-810, thereby transforming the computer system 1200 in FIG. 8 to a special purpose machine for implementing the operations described herein. Examples of the computer system 800 include personal computers, terminals, workstations, mobile phones, tablets, laptops, personal computers, multimedia consoles, gaming consoles, set top boxes, and the like.

The one or more data storage devices 804 may include any non-volatile data storage device capable of storing data generated or employed within the computing system 800, such as computer executable instructions for performing a computer process, which may include instructions of both application programs and an operating system (OS) that manages the various components of the computing system 800. The data storage devices 804 may include, without limitation, magnetic disk drives, optical disk drives, solid state drives (SSDs), flash drives, and the like. The data storage devices 1804 may include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, SSDs, and the like. The one or more memory devices 1206 may include volatile memory (e.g., dynamic random access memory (DRAM), static random access memory (SRAM), etc.) and/or non-volatile memory (e.g., read-only memory (ROM), flash memory, etc.).

Computer program products containing mechanisms to effectuate the systems and methods in accordance with the presently described technology may reside in the data storage devices 804 and/or the memory devices 806, which may be referred to as machine-readable media. It will be appreciated that machine-readable media may include any tangible non-transitory medium that is capable of storing or encoding instructions to perform any one or more of the operations of the present disclosure for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures.

In some implementations, the computer system 800 includes one or more ports, such as an input/output (I/O) port 1808 and a communication port 810, for communicating with other computing, network, or vehicle devices. It will be appreciated that the ports 808-810 may be combined or separate and that more or fewer ports may be included in the computer system 1200.

The I/O port 808 may be connected to an I/O device, or other device, by which information is input to or output from the computing system 800. Such I/O devices may include, without limitation, one or more input devices, output devices, and/or environment transducer devices.

In one implementation, the input devices convert a human-generated signal, such as, human voice, physical movement, physical touch or pressure, and/or the like, into electrical signals as input data into the computing system 800 via the I/O port 808. Similarly, the output devices may convert electrical signals received from computing system 800 via the I/O port 808 into signals that may be sensed as output by a human, such as sound, light, and/or touch. The input device may be an alphanumeric input device, including alphanumeric and other keys for communicating information and/or command selections to the processor 802 via the I/O port 808. The input device may be another type of user input device including, but not limited to: direction and selection control devices, such as a mouse, a trackball, cursor direction keys, a joystick, and/or a wheel; one or more sensors, such as a camera, a microphone, a positional sensor, an orientation sensor, a gravitational sensor, an inertial sensor, and/or an accelerometer; and/or a touch-sensitive display screen (“touchscreen”). The output devices may include, without limitation, a display, a touchscreen, a speaker, a tactile and/or haptic output device, and/or the like. In some implementations, the input device and the output device may be the same device, for example, in the case of a touchscreen.

The environment transducer devices convert one form of energy or signal into another for input into or output from the computing system 800 via the I/O port 808. For example, an electrical signal generated within the computing system 800 may be converted to another type of signal, and/or vice-versa. In one implementation, the environment transducer devices sense characteristics or aspects of an environment local to or remote from the computing device 800, such as, light, sound, temperature, pressure, magnetic field, electric field, chemical properties, physical movement, orientation, acceleration, gravity, and/or the like. Further, the environment transducer devices may generate signals to impose some effect on the environment either local to or remote from the example computing device 800, such as, physical movement of some object (e.g., a mechanical actuator), heating or cooling of a substance, adding a chemical substance, and/or the like.

In one implementation, a communication port 810 is connected to a network by way of which the computer system 1800 may receive network data useful in executing the methods and systems set out herein as well as transmitting information and network configuration changes determined thereby. Stated differently, the communication port 810 connects the computer system 800 to one or more communication interface devices configured to transmit and/or receive information between the computing system 800 and other devices by way of one or more wired or wireless communication networks or connections. Examples of such networks or connections include, without limitation, Universal Serial Bus (USB), Ethernet, Wi-Fi, Bluetooth®, Near Field Communication (NFC), Long-Term Evolution (LTE), and so on. One or more such communication interface devices may be utilized via the communication port 1210 to communicate one or more other machines, either directly over a point-to-point communication path, over a wide area network (WAN) (e.g., the Internet), over a local area network (LAN), over a cellular (e.g., third generation (3G) or fourth generation (4G)) network, or over another communication means. Further, the communication port 810 may communicate with an antenna or other link for electromagnetic signal transmission and/or reception.

In an example implementation, pulmonary health management data, air analytics, and software and other modules and services may be embodied by instructions stored on the data storage devices 804 and/or the memory devices 806 and executed by the processor 802. The computer system 800 may be integrated with or otherwise form part of the inhalation device 100.

The system set forth in FIG. 8 is but one possible example of a computer system that may employ or be configured in accordance with aspects of the present disclosure. It will be appreciated that other non-transitory tangible computer-readable storage media storing computer-executable instructions for implementing the presently disclosed technology on a computing system may be utilized.

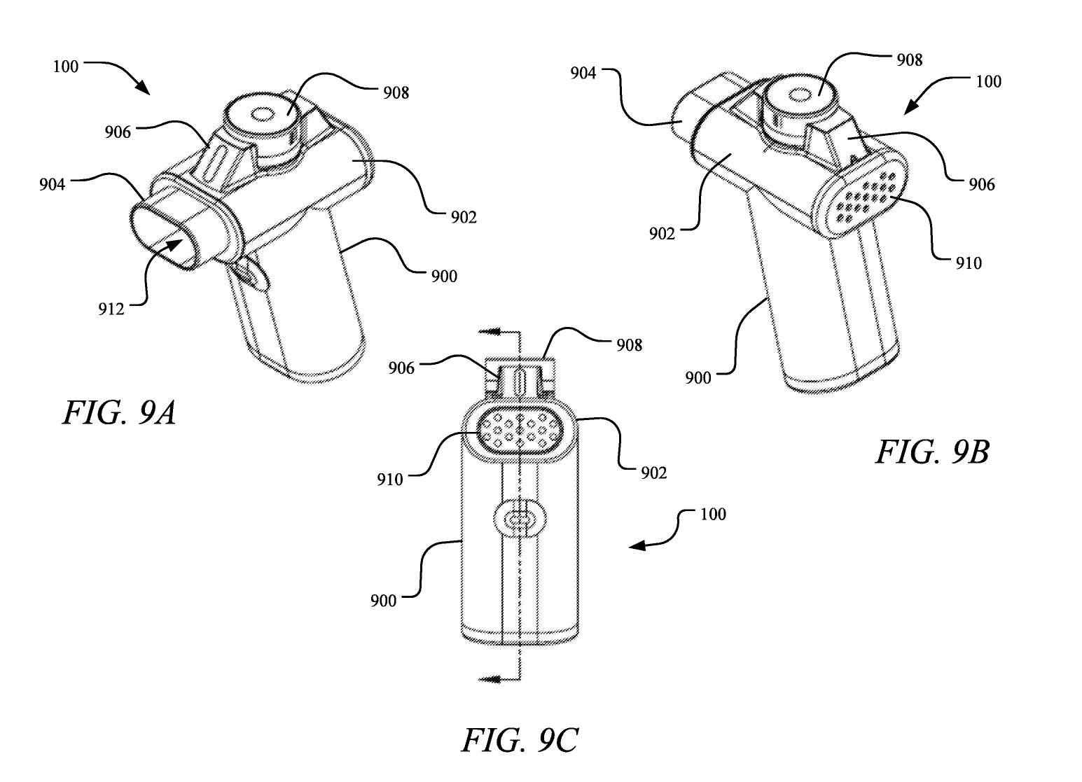

FIGS. 9A-16K provide specific examples of various aspects of the inhalation device 100. The description of these aspects is exemplary only and not intended to be limiting.

Turning to FIGS. 9A-9C, in one implementation, the inhalation device 100 includes a handle 900, a housing 902 enclosing a mouthpiece 904, a cartridge mount 906 for receiving a disposable or reusable cartridge 908, a laminar flow element 910, and an opening 912 defined by the mouthpiece 904.

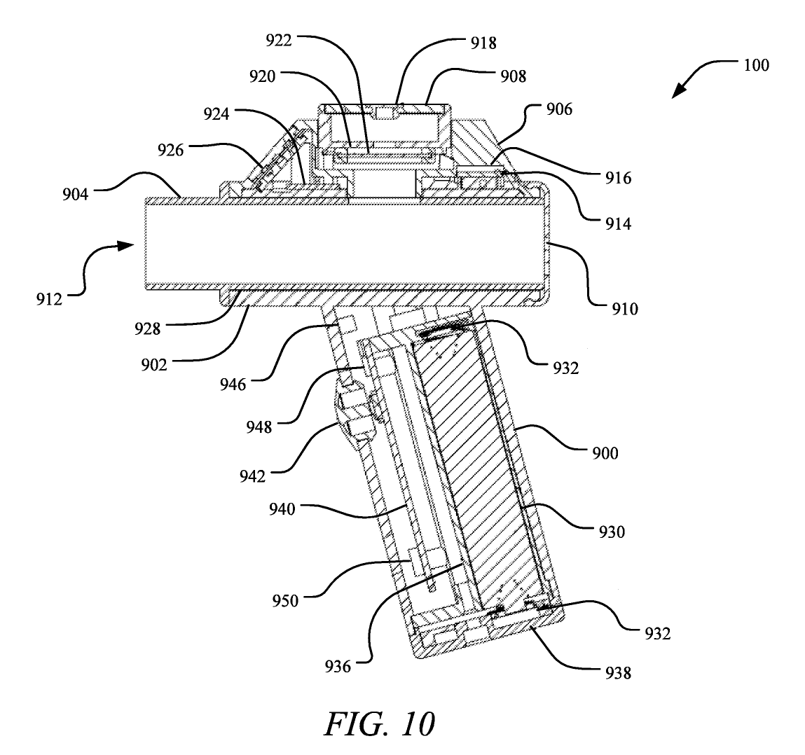

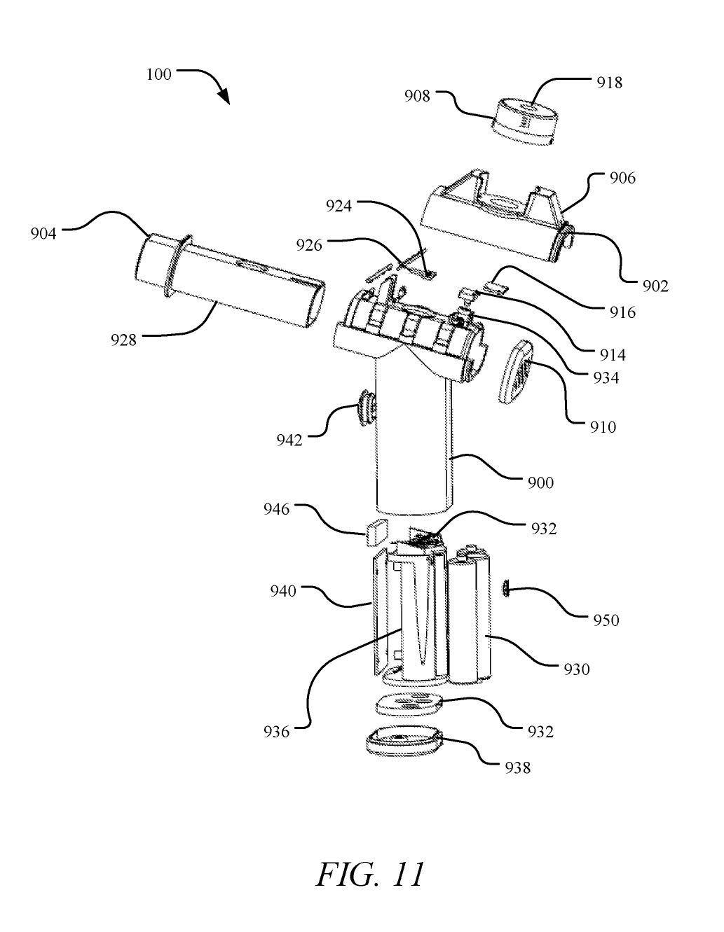

FIGS. 10 and 11 illustrates a cross-section and exploded view, respectively, of the inhalation device 100 shown in FIGS. 9A-9C. In one implementation, the inhalation device 100 includes a pressure sensor assembly 914, a pressure sensor electronics board 916, a syperhydrophobic valve 918, a piezoelectric actuator 920, an aperature plate 922, a light source electronics board 924, one or more light sources 926 (e.g., LEDs), a aerosol tube 928 communicating with the mouthpiece 904, a power source 930, power source contacts 932, a pressure sensor o-ring 934, a power source mount 936, a removable handle cover 938, a controller 940, a manual dispense button 942, a speaker 946, an audio chip 948, and a user device link 950. These components may be used to perform one or more of the operations discussed herein.

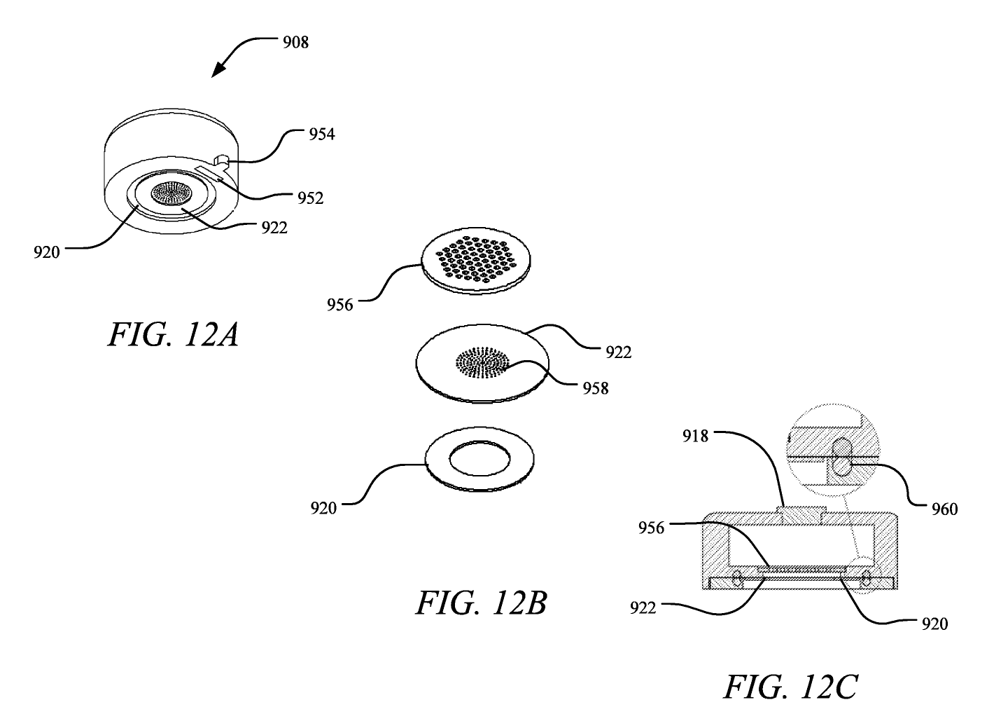

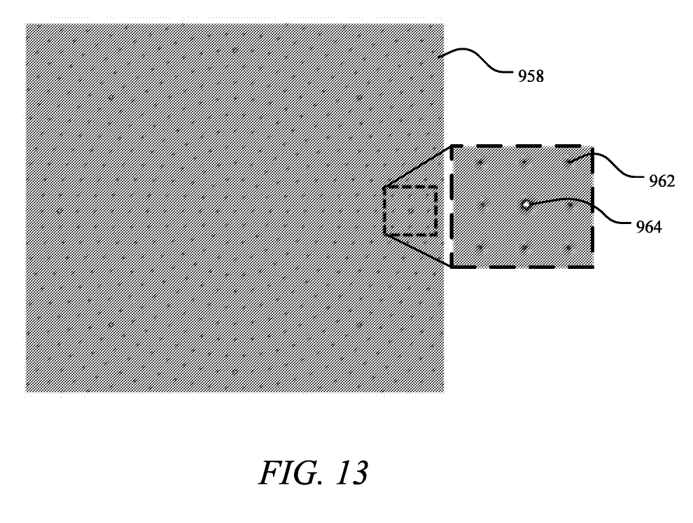

As shown in FIGS. 12A-12C, the cartridge 908 may include a guide 954 and a cartridge tag 952. The ejector assembly of the cartridge 908 may include a surface tension plate 956, the actuator 920, an ejector o-ring 960, and the aperature plate 922, which may have droplet forming openings 958. As shown in FIG. 13, the droplet forming openings 958 may be a plurality of sizes (e.g., 962 and 964) to generate different droplet sizes for targeting specific regions of the pulmonary airways.

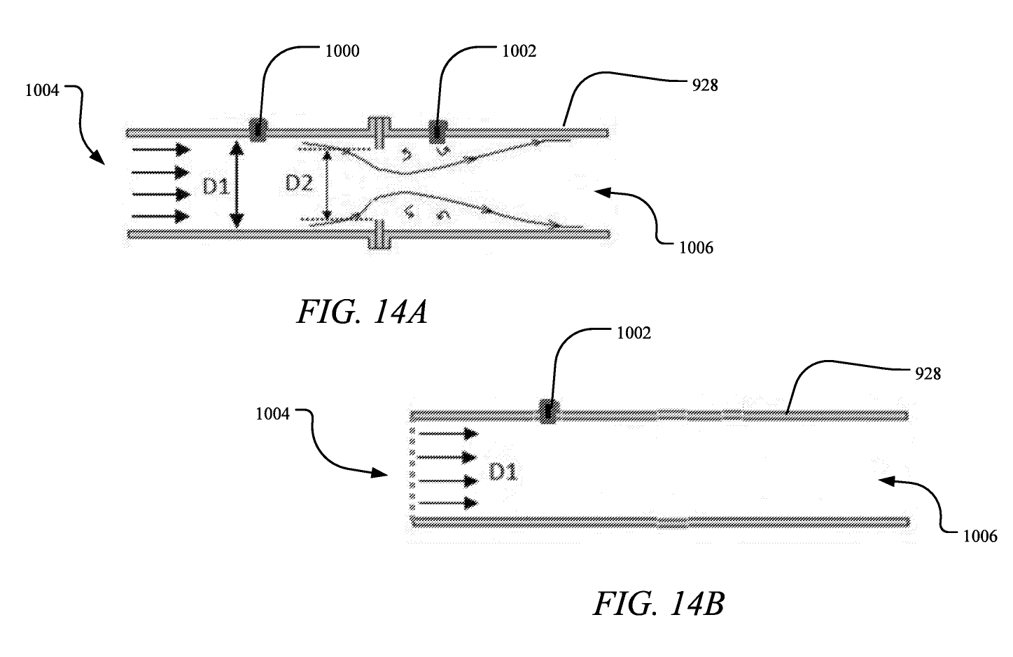

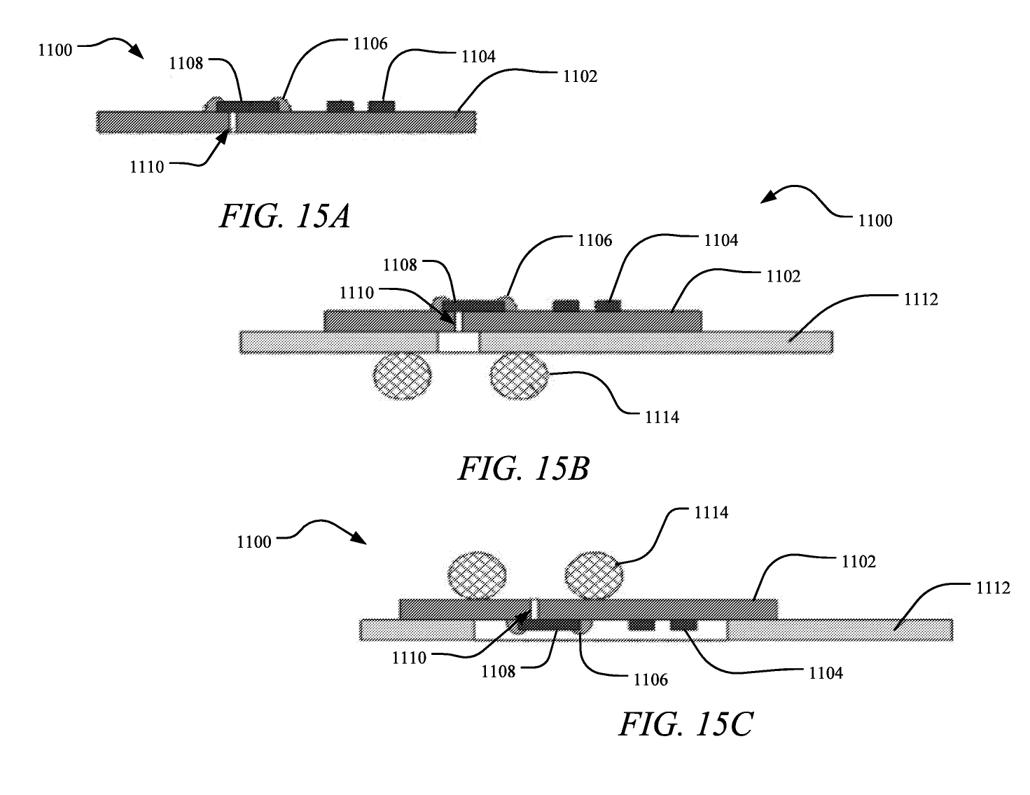

FIGS. 14A-15C illustrates various configurations of the pressure sensor assembly 914. In one implementation shown in FIG. 14A, the pressure sensor assembly 914 includes a first pressure sensor 1000, a second pressure sensor 1002 to sense airflow by detecting pressure differentials across air flow restriction internal to the aerosol tube 928. FIG. 14B shows the pressure sensor assembly 914 with one pressure sensor 1002 where the restriction is the laminar flow screen 910 and the pressure is sensed as the differential between the interior of the tube 928 and the pressure outside the tube 928. The first and second pressure sensors 1000 and 1002 may be used for spray verification by detecting pressure differentials between the interior and exterior areas of the aerosol tube 928 as an airflow moves from an inlet 1004 to an outlet 1006. The pressure sensor assembly 914 may further coordinate a breathing cycle for the patient 104 to identify a trigger point to activate a spray during a peak period during an inhalation cycle.

FIG. 14A shows the pressure sensor assembly 914 having one pressure sensor 1000 sensing pressure inside the tube 928 and the second sensor 1002 sensing atmospheric pressure outside the tube. This configuration allows a very low airflow internal resistance. The very low internal resistance for a droplet delivery device has great benefits. Since the device 100 is breath actuated for spray and delivering of medication in the form of aerosols, the very low internal resistance to airflow provides patients with the opportunity for deep inhalation and therefore allows the aerosol particles to reach deep into the pulmonary airways. The breath actuated triggering mechanism for delivering an aerosol dose further provides assurance for optimum delivery of medication.

FIGS. 15A-15C show examples of the delta P sensor assembly 1100 of the sensor electronics 916 and pressure sensor assembly 914 having a printed circuit board (PCB) 1102, a pressure sensor controller 1104, a sensor die 1108, a glob-top 1106, and a hole 110. The assembly 1100 may further include a mother board 1112 and o-ring 1114. The example include a one port design and its assembly to the device board (15A), the sensor having a pneumatic connection through the hole in the PCB and mounted on the main PCB or a daughter board as shown on schemes in (F15B) or (15C).

The delta P sensor specifications, not limited by example, may follow:

Pressure range; +/−500 Pa range; delta P measurement relative to ambient;

Absolute accuracy; 3% or better

Repeatability; 3% or better

FS resolution; 1% or better

Digital or analog output; Digital preferred; I²C or SPI

Supply voltage; 3 VDC+/−5%

Offset, auto zeroing; after turning on;

Storage temperature; −20 to +60 C;

Operating temperature 10 to 45 C;

The sensor assembly 914 may outperform traditional piezoresistive membrane sensors in terms of sensitivity at low differential pressure, offset drift, and hysteresis. These performance parameters allow increased resolution and more accurate measurements at low pressures. These performance parameters are particularly important for children and elderly patients with low pulmonary output.

In on implementation, the signal generated by the pressure sensors provides a trigger for activation of a spray at or during a peak period of an inhalation (inspiratory) cycle of the patient 104 and assures optimum deposition of the aerosol spray and delivery of the medication into the pulmonary airways, as described herein.

In addition, an image capture device, including cameras, scanners, or other sensors without limitation, e.g. charge coupled device (CCD), may be provided to detect and measure the ejected aerosol plume. These detectors, LED, delta P transducer, CCD device, all provide controlling signals to the controller 940 for monitoring, sensing, measuring and controlling the ejection of fluid and reporting patient compliance, treatment times, dosage, and patient usage history, etc., which may be communicated to the user device 204 via the link 950.

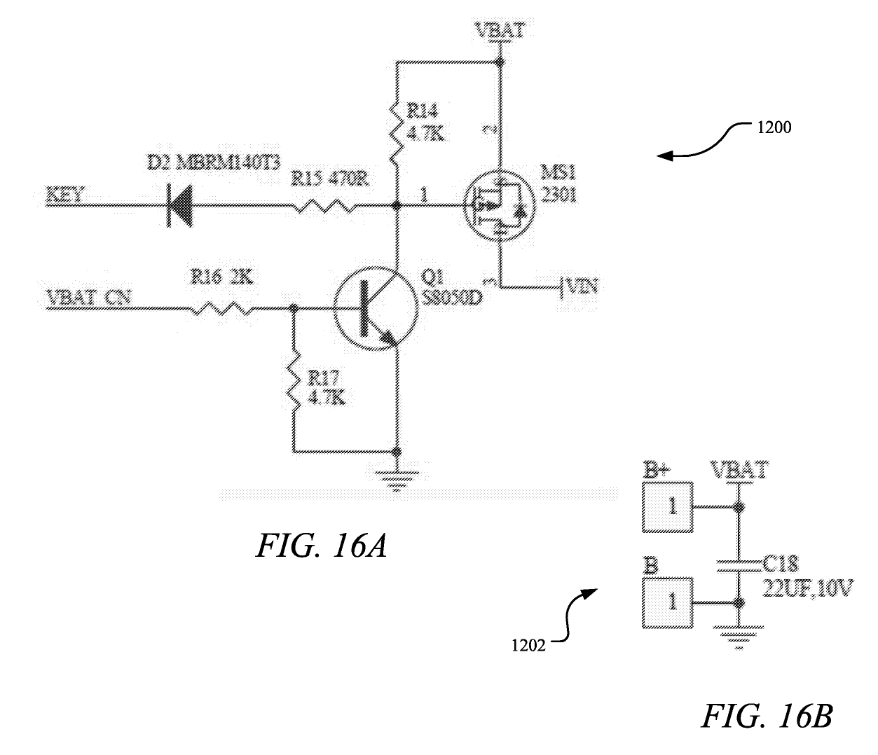

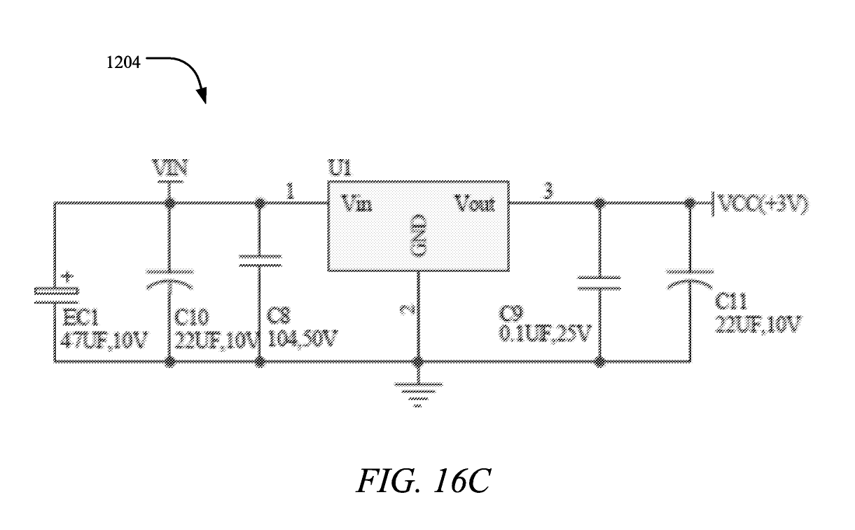

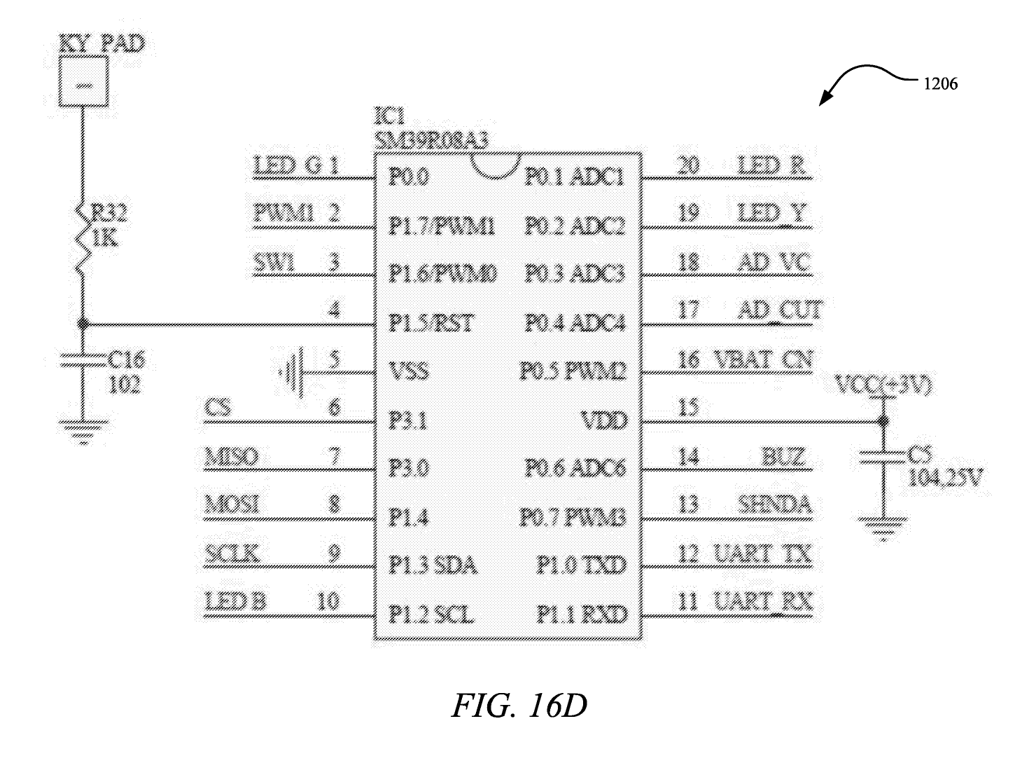

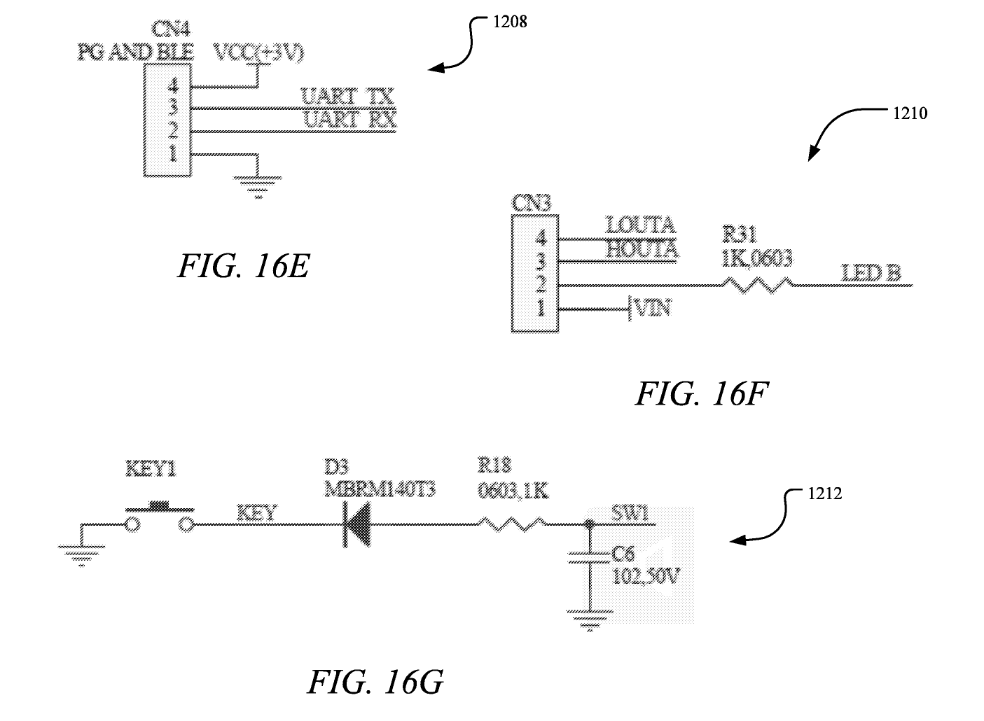

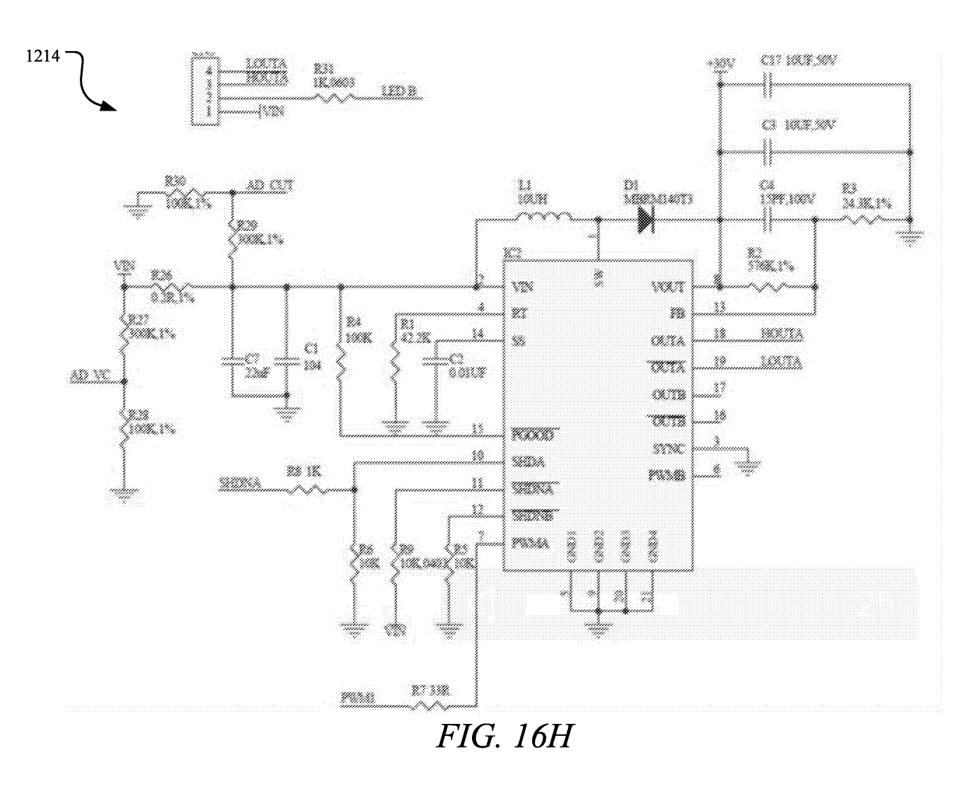

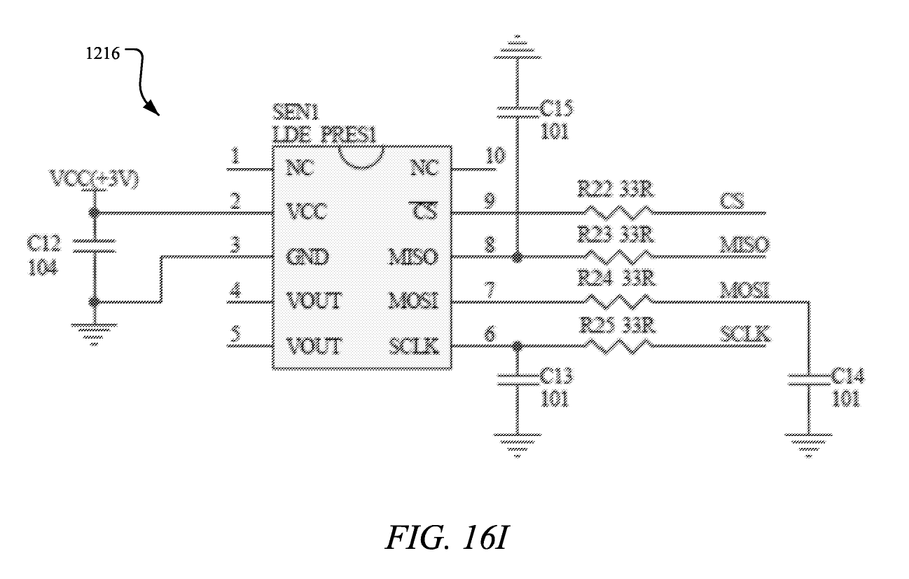

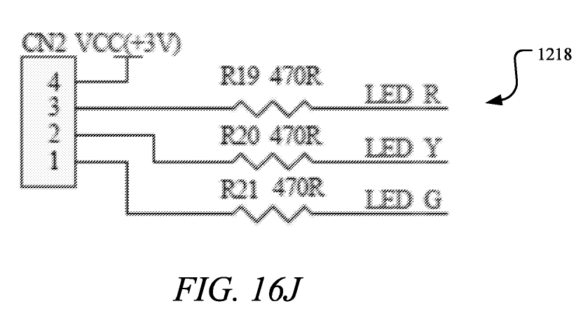

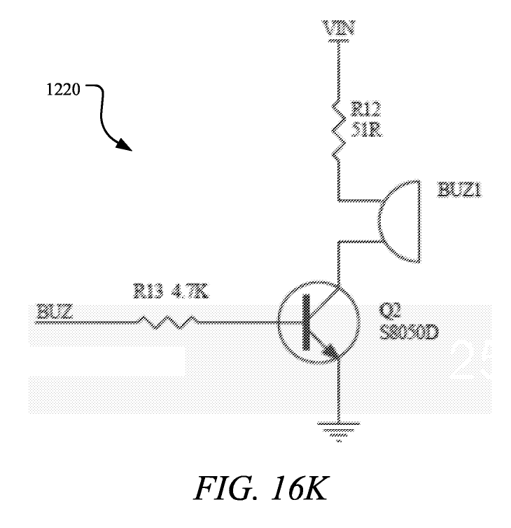

Turning to FIGS. 16A-16K, various example circuit diagrams 1200-1220 are provided that are exemplary only and not intended to be liming. The circuit diagrams include a power supply on/off circuit 1200, a power supply connector circuit 1202, a volt power conditioning circuit 1204, a controller circuit 1206, a controller programming connector circuit 1208, a piezo connector circuit 1210, a user switch with debounce circuit 1212, a piezo driver circuit 1214, a pressure sensor circuit 1216, a light emitting diode indicator circuit 1218, and a buzzer driver circuit 1220. Variations to these circuits and addition of, deletion of, or other modifications to the circuits are contemplated.

In the present disclosure, the methods disclosed may be implemented as sets of instructions or software readable by a device. Further, it is understood that the specific order or hierarchy of steps in the methods disclosed are instances of example approaches. Based upon design preferences, it is understood that the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a non-transitory machine-readable medium having stored thereon instructions, which may be used to program a computer system (or other electronic devices) to perform a process according to the present disclosure. A machine-readable medium includes any mechanism for storing information in a form (e.g., software, processing application) readable by a machine (e.g., a computer). The machine-readable medium may include, but is not limited to, magnetic storage medium, optical storage medium; magneto-optical storage medium, read only memory (ROM); random access memory (RAM); erasable programmable memory (e.g., EPROM and EEPROM); flash memory; or other types of medium suitable for storing electronic instructions.

While the present disclosure has been described with reference to various implementations, it will be understood that these implementations are illustrative and that the scope of the present disclosure is not limited to them. Many variations, modifications, additions, and improvements are possible. More generally, embodiments in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. A method for pulmonary health management, the method comprising:
receiving pulmonary health management information from one or more inhalation devices over a network, each of the one or more inhalation devices having one or more pressure sensors measuring a flow rate of an air flow through a tube of the inhalation device;
receiving environmental data for one or more geographical locations in which the one or more inhalation devices are deployed, the environmental data captured using one or more environmental sensors and corresponding to an ambient air condition of each of the one or more geographical locations;
correlating the pulmonary health management information with the environmental data based on at least one management parameter using at least one computing unit; and
generating air analytics from the correlated data using the at least one computing unit.

2. The method of claim 1, further comprising: outputting the air analytics for display on a user device.

3. The method of claim 2, wherein the user device is at least one of a patient device or a provider device.

4. The method of claim 1, wherein the at least one management parameter includes at least one of: disease type, a patient profile, spray verification types, environmental condition type, device error type, or pollutant type.

5. The method of claim 1, wherein the air analytics includes at least one of: air quality analytics, disease analytics, healthcare analytics, device analytics, alerts, or trends.

6. A method for pulmonary health management for a patient, the method comprising:
receiving a peak inspiratory flow measurement achieved during inhalation by the patient, the peak inspiratory flow measurement generated based on a minimum pressure of an air flow during the inhalation;
receiving a pulmonary output for the patient, the pulmonary output determined by measuring a maximum flow achieved during an exhalation;
generating pulmonary health management information for the patient using at least one computing unit, the pulmonary health management information including a pulmonary health profile generated based on the peak inspiratory flow measurement and the pulmonary output; and
outputting the pulmonary health management information.

7. The method of claim 6, wherein the pulmonary health profile is further generated based on a concentration value of nitric oxide in the exhalation.

8. The method of claim 6, wherein the pulmonary health management information is output for presentation with a user device.

9. The method of claim 6, wherein the pulmonary health profile is further generated based on an airway resistance.

10. The method of claim 6, wherein the pulmonary health management information is communicated by a user device to an air analyzer over a network.

11. The method of claim 6, further comprising: tracking a change in the pulmonary health management information over time.

12. The method of claim 6, wherein the pulmonary health profile includes at least one of a diagnosis of a pulmonary condition or a treatment for the pulmonary condition.

13. A method for pulmonary health management for a patient, the method comprising:
identifying an initiation of an inhalation cycle by the patient;
determining a trigger point in the inhalation cycle by measuring a flow rate of an air flow using one or more pressure sensors;
spraying an aerosol plume into the air flow;
generating an ejection of a set of droplets automatically into the aerosol plume at the trigger point using an ejector assembly; and
validating the ejection of the set of droplets.

14. The method of claim 13, wherein the one or more pressure sensors include a first sensor disposed upstream in the air flow and a second sensor disposed external to the air flow, the first sensor measuring an internal pressure and the second sensor measuring an external pressure, the flow rate measured from a pressure differential between the internal pressure and the external pressure.

15. The method of claim 13, wherein the flow rate is measured based on a pressure drop between the air flow and a surrounding atmosphere.

16. The method of claim 13, wherein the set of droplets includes one or more droplets each having a size of five microns or less.

17. The method of claim 13, wherein the trigger point corresponds to a peak in the inhalation cycle.

18. The method of claim 13, wherein the ejection of the set of droplets is validated by detecting a velocity of an ejection mass in the aerosol plume.

19. The method of claim 13, wherein the ejection of the set of droplets is validated by detecting a cross-section and a length of the aerosol plume.

20. The method of claim 13, wherein the trigger point is preset.

21. The method of claim 13, further comprising: communicating validation information to an air analyzer using a user device, the validation information generated based on the validation of the ejection of the set of droplets.

22. The method of claim 21, further comprising: generating feedback based on the validation information.
